(19) United States
(12) Patent Application Publication
(10) Pub. No.: US 2024/0075150 A1
(43) Pub. Date: Mar. 7, 2024

(54) CONJUGATES COMPRISING REVERSIBLE LINKERS AND USES THEREOF

(71) Applicant: Ascendis Pharma A/S, Hellerup (DK)

(72) Inventors: Samuel WEISBROD, Heidelberg (DE); Roxana LEMNARU, Heidelberg (DE)

(21) Appl. No.: 18/105,460

(22) Filed: Feb. 3, 2023

Related U.S. Application Data
(63) Continuation of application No. PCT/EP2021/071775, filed on Aug. 4, 2021.

(30) Foreign Application Priority Data
Aug. 5, 2020 (EP) 20189583.6
Dec. 21, 2020 (EP) 20216058.6

(51) Int. Cl. A61K 47/60; A61K 47/54

(52) U.S. Cl. CPC A61K 47/60; A61K 47/545; A61K 47/549

(57) ABSTRACT

The present invention relates to conjugates comprising reversible linkers and pharmaceutically acceptable salts thereof, pharmaceutical compositions comprising said conjugates and the use of said conjugates as medicaments.

The present invention relates to conjugates comprising reversible linkers and pharmaceutically acceptable salts thereof, pharmaceutical compositions comprising said conjugates and the use of said conjugates as medicaments.

To improve physicochemical or pharmacokinetic properties, such as the in vivo circulation half-life of drugs, such drugs can be conjugated to a carrier, such as a polymer. Typically, polymers in drug delivery are either used in a non-covalent complexation of the drug and polymer, embedding of drug in a polymer or by covalent conjugation of the drug to a polymeric moiety.

However, the non-covalent approach requires a highly efficient drug encapsulation to prevent uncontrolled, burst-type release of the drug due to the disintegration of the drug-polymer complex after administration. Restraining the diffusion of an unbound, water-soluble drug molecule requires strong van der Waals contacts, frequently mediated through hydrophobic moieties and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a polymeric moiety via a stable linker or a reversible linker from which the drug is released. If the drug is stably conjugated to the polymeric moiety, such conjugate needs to exhibit sufficient residual activity to have a pharmaceutical effect and thus the conjugate is constantly in an active form.

One advantage of conjugating a drug to a polymeric moiety through a reversible linker is that no residual activity of the conjugate is needed, because the drug exhibits its pharmacological effect upon release from the conjugate. A conjugate may exhibit no or little drug activity, i.e. the conjugate is pharmacologically inactive. This approach is applied to all classes of molecules, from so-called small molecules, through natural products up to large proteins. The drug of such a conjugate may be released by enzymatic or non-enzymatic cleavage of the linkage between the polymeric moiety and the drug moiety or by a combination of both.

However, enzyme-dependence is usually less preferred because enzyme levels may vary significantly between patients what makes the correct dosing difficult.

It is thus an object of the present invention to at least partially overcome the shortcomings described above.

This object is achieved with a conjugate or a pharmaceutically acceptable salt thereof comprising at least one moiety -D, conjugated via at least one moiety -L¹-L²- to at least one moiety Z, wherein a moiety -L¹- is conjugated to a moiety -D and wherein the linkage between -D and -L¹- is reversible and wherein a moiety -L²- is conjugated to Z, wherein
each -D is independently a drug moiety;
each -L²- is independently a single bond or a spacer moiety;
each Z is independently a polymeric moiety or a C₈₋₂₄ alkyl;
each -L¹- is independently a linker moiety of formula (I):

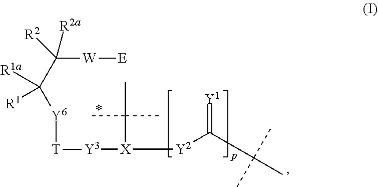

wherein
the dashed line marked with an asterisk indicates the attachment to -L²-;
the unmarked dashed line indicates the attachment to -D;
p is selected from the group consisting of 0 and 1;
—W— is selected from the group consisting of —O— and —N(R³)—;
-E is a cleavable moiety;
-T- is selected from the group consisting of

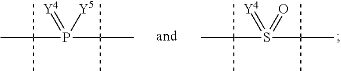

═Y¹ and ═Y⁴ are independently selected from the group consisting of ═O, ═S and ═N(R⁴);
—Y²—, —Y³— and —Y⁶— are independently selected from the group consisting of —O—,
—S— and —N(R⁴)—;
—Y⁵ is selected from the group consisting of —O(R⁵), —S(R⁵) and —N(R⁴)(R^{4a});
—X— is selected from the group consisting of:
C₂₋₃ alkyl,

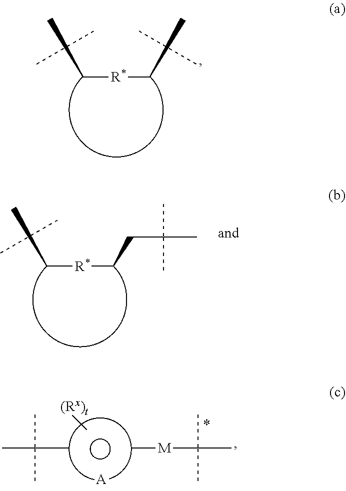

provided that if —X— is C₂₋₃ alkyl, of formula (a) or (b) then p is 1;
—R*— is selected from the group consisting of C₃₋₁₀ cycloalkyl and 3- to 10-membered heterocyclyl;
wherein in formula (c) the unmarked dashed line indicates the attachment point to —Y³— and the marked dashed line indicates the attachment to —Y²— if p is 1 or to -D if p is 0 and wherein -A- is a ring selected from the group consisting of mono-, bi- or tricylic aryl and heteroaryl, provided that -A- is connected to —Y³- and -M- via carbon atoms; wherein said mono-, bi- or tricyclic aryl and heteroaryl is optionally substituted with one or more —R^{x} which are the same or different;
t is selected from the group consisting of 0, 1, 2, 3, 4, 5, 6, 7, 8, 9 and 10; wherein -M- is selected from the group consisting of formula (i) and (ii):

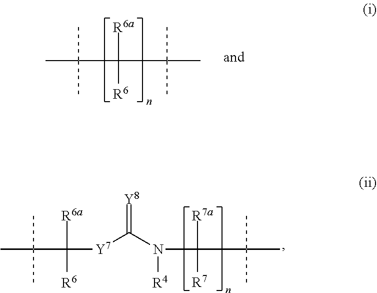

—Y⁷— is selected from the group consisting of —O—, —S— and —N(R⁴)—;
═Y⁸ is selected from the group consisting of ═O, ═S and ═N(R⁴);
n is independently selected from the group consisting of 1, 2 and 3;
—R¹, —R^{1a}, —R², —R^{2a} are independently selected from the group consisting of —H, —C(O)OH, halogen, —NO₂, —CN, —OH, -T*, C₁₋₆ alkyl, C₂₋₆ alkenyl and C₂₋₆ alkynyl;
wherein C₁₋₆ alkyl, C₂₋₆ alkenyl and C₂₋₆ alkynyl are optionally substituted with one or more —R^{x}, which are the same or different; and wherein C₁₋₆ alkyl, C₂₋₆ alkenyl and C₂₋₆ alkynyl are optionally interrupted by one or more groups selected from the group consisting of -T*-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
—R⁵, each —R⁶, each —R^{6a}, each —R⁷ and each —R^{7a} are independently selected from the group consisting of —H, -T*, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{x}, which are the same or different; and wherein C₁₋₆ alkyl, C₂₋₆ alkenyl and C₂₋₆ alkynyl are optionally interrupted by one or more groups selected from the group consisting of -T*-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each T* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T* is independently optionally substituted with one or more —R^{x}, which are the same or different;
   each —R^{x} is independently selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
wherein —R³, —R⁴ and —R^{4a} are independently selected from the group consisting of —H and C₁₋₆ alkyl; wherein C₁₋₆ alkyl is optionally substituted with one or more halogen, which are the same or different;
optionally, —R¹ and —R² are joined together to form a carbohydrate-nucleobase system;
optionally, —R¹ and —R² are joined together with the carbon atoms to which they are attached to form a ring T*;
optionally, —R³ and —R² together with the nitrogen to which —R³ is attached and the carbon atom to which —R² is attached form a 3- to 10-membered heterocyclyl or an 8- to 11-membered heterobicyclyl, which 3- to 10-membered heterocyclyl or 8- to 11-membered heterobicyclyl is optionally substituted with one or more —R^{x} which are the same or different;
optionally, —R³ and —R¹ together with the nitrogen to which —R³ is attached, the carbon atom to which —R² is attached and the carbon atom to which —R¹ is attached form a 3- to 10-membered heterocyclyl or an 8- to 11-membered heterobicyclyl, which 3- to 10-membered heterocyclyl or 8- to 11-membered heterobicyclyl is
optionally substituted with one or more —R^{x} which are the same or different; optionally, —R⁶ and an adjacent —R^{6a} of formula (i) or —R⁷ and an adjacent —R⁷ of formula (ii) form a carbon-carbon double bond provided that n is selected from the group consisting of 2 and 3; and
wherein each -L¹- is substituted with -L²- and optionally further substituted.

It is understood that if —X— is C₂₋₃ alkyl, then -L²- can be attached at any of the carbon atoms such as for example by substituting one of the hydrogen atoms of C₂₋₃ alkyl with -L²-. Similarly, it is understood that if —X— is of formula (a), then -L²- can be attached at any of the atoms on the ring —R*— of formula (a) and if —X— is of formula (b) then -L²- can be attached at any of the atoms on the ring —R*— of formula (b) or on the methylene carbon that is not part of the ring —R*— of formula (b), such as by substituting one of the hydrogen atoms on —R*— of formula (a) or (b) or on the methylene of formula (b) with -L²-. Also, it is understood that if —X— is of formula (c), then -L²- can be attached at any of the atoms either of -A- or -M-, such as for example by substituting one of the hydrogen atoms of -A- or -M- with -L²-.

The conjugates disclosed herein are capable of releasing the drug in an unmodified form through intramolecular cyclization reaction or may undergo a 1,(4+2p′) elimination reaction wherein p′ is for example 0, 1 or 2. More details regarding such release mechanism can be found for example in WO18163131A1, WO19171358A1 and WO05099768A2 which are incorporated herein by reference. One difference between the conjugates described in WO18163131A1, WO19171358A1 or WO05099768A2 which may undergo the 1,(4+2p′) elimination and the conjugates of the present invention is the position of the moiety Z. Within the conjugates of WO18163131A1, WO19171358A1 or WO05099768A2, the aliphatic polymeric moiety is attached to the phosphate group, whereas within the conjugates of the present invention Z is attached on the ring -A- or on -M-. Applicant surprisingly found that by attaching the Z moiety on the ring -A- or on -M- the release of potentially toxic side-products such as quinone methide is avoided, as upon the 1,(4+2p′) elimination quinone methide remains covalently attached to Z. This means that the conjugates of the present invention overcome some of the disadvantages of the conjugates of WO18163131A1, WO19171358A1 and WO05099768A2. This is significant as these potentially toxic entities are released in a 1:1 stoichiometry with the drug and can accumulate in high in vivo concentrations. Applicant also surprisingly found that in order to ensure that the drug is released in its unmodified form from the conjugates or pharmaceutically acceptable salt thereof of the present invention, it is desirable that Z is located in closer proximity to the drug moiety, such as by being attached to —X— of formula (I) via -L²-.

Within the meaning of the present invention the terms are used as follows.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety is released from a reversible linkage as a drug.

As used herein, the term “carbohydrate-nucleobase system” refers to a ring system comprising a carbohydrate such as for example a substituted ribofuranose, arabinofuranose, xylofuranose or lyxofuranose and a nucleobase such as for example adenosine, guanosine, 5-methyluridine, uridine, 5-methylcytidine, cytidine, inosine, xanthosine or wybutosine and having the following general structure:

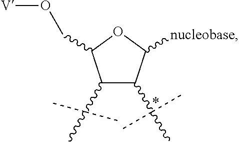

wherein the dashed line marked with an asterisk indicates the attachment to —W—of formula (I) and the unmarked dashed line indicates the attachment to —Y⁶— of formula (I) or vice versa; and
—V′ is selected from the group consisting of —H and protecting group.

As used herein, the term “cleavable moiety” is a moiety which can be cleaved or transformed by a chemical, photochemical or biological process such as by an enzymatic process under acidic or basic conditions such as physiological conditions which are aqueous buffer at pH 7.4 and 37° C.

As used herein, the term “protecting group” refers to a moiety that is used for the reversible protection of functional groups during chemical reaction processes to render these functional groups unreactive in said chemical reaction processes. Suitable protecting groups are well known in the art.

It is understood that if a sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” may be attached to two moieties or interrupting a moiety either as “—C(O)N(R^{x})—” or as “—N(R^{x})C(O)—”. Similarly, a moiety:

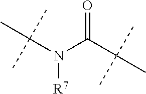

may be attached to two moieties or may interrupt a moiety either as:

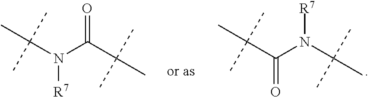

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the conjugates of the present invention are prodrugs. As used herein, the term “prodrug” refers to a drug moiety, that is reversibly and covalently conjugated to, for example, a polymeric moiety, such as Z, through at least one -L¹-L²- moiety. A prodrug releases the reversibly and covalently bound drug moiety -D in the form of its corresponding drug D-H. In other words, a prodrug is a conjugate comprising a drug moiety, which is covalently and reversibly conjugated to a, for example, polymeric moiety via at least one -L¹-L²- moiety. Such prodrugs or conjugates release the formerly conjugated drug moiety in the form of a free drug.

As used herein, the term “reversible linkage” or “biodegradable linkage” is a linkage that is cleavable, in the absence of enzymes under physiological conditions, which are aqueous buffer at pH 7.4 and 37° C., with a half-life ranging from one hour to six months, such as from one hour to four months, such as from one hour to three months, from one hour to two months or from one hour to one month. It is understood, however, that a reversible linkage may also be cleavable at other conditions, such as for example at a different pH or at a different temperature with a half-life ranging from one hour to six months, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “stable” and “stability” with regards to a pharmaceutical formulation or composition comprising a conjugate of the present invention means that after a storage time, such as after one month, two months, four months, six months, eight months, twelve months, eighteen months, twenty-four months, thirty-six months, in particular after the indicated storage time, the pharmaceutical formulation or composition comprises less than 5% of the drug in its free form.

As used herein, the term “reversible linker moiety” is a moiety which is covalently conjugated to a drug moiety through a reversible linkage and which is also covalently conjugated to a moiety Z via a moiety -L²-. In certain embodiments, the linkage between Z and -L²- is a stable linkage.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₈₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

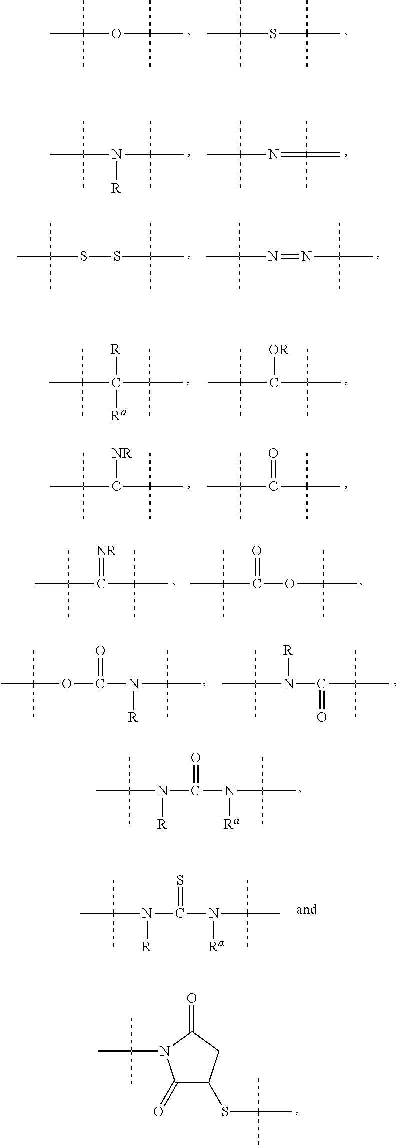

wherein
dashed lines indicate attachment to the remainder of the moiety or reagent;
—R and —Ra are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

As used herein, the term “monocyclic, bicyclic or tricyclic aryl” means an aromatic hydrocarbon ring system which may be monocyclic, bicyclic or tricyclic, wherein the monocyclic aryl ring consists of at least 5 ring carbon atoms and may comprise up to 10 ring carbon atoms, wherein the bicylic aryl ring consists of at least 8 ring carbon atoms and may comprise up to 12 ring carbon atoms and wherein the tricylic aryl ring consists of at least 12 ring carbon atoms and may comprise up to 14 ring carbon atoms. Each hydrogen atom of a monocyclic, bicyclic or tricyclic aryl may be replaced by a substituent as defined below.

As used herein, the term “monocyclic, bicyclic or tricyclic heteroaryl” means a monocyclic aromatic ring system that may comprise 2 to 6 ring carbon atoms and 1 to 3 ring heteroatoms, a bicyclic aromatic ring system that may comprise 3 to 9 ring carbon atoms and 1 to 5 ring heteroatoms or a tricyclic aromatic ring system that may comprise 7 to 13 ring carbon atoms and 1 to 6 ring heteroatoms, such as nitrogen, oxygen and sulfur. Examples for monocyclic, bicyclic or tricyclic heteroaryl groups include, but are not limited to, benzofuranyl, benzothiophenyl, furanyl, imidazolyl, indolyl, azaindolyl, azabenzimidazolyl, benzoxazolyl, benzothiazolyl, benzothiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl, thiophenyl, dibenzopyrrole, dibenzofuran and xanthene. Each hydrogen atom of a monocyclic, bicyclic or tricyclic heteroaryl may be replaced by a substituent as defined below.

As used herein, the term “nucleophile” refers to a reagent or functional group that forms a bond to its reaction partner, i.e. the electrophile by donating both bonding electrons.

It is understood that the phrase “—R^{x} and —R^{y} are joined together with the carbon atoms to which they are attached to form a ring T*” in relation with a moiety of the structure:

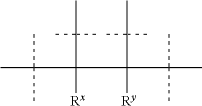

means that —R^{x} and —R^{y} form the following structure:

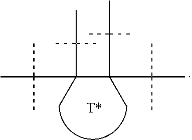

It is also understood that the phrase “—R³ and —R² together with the nitrogen to which —R³ is attached and the carbon atom to which —R² is attached form a 3- to 10-membered heterocyclyl or an 8- to 11-membered heterobicyclyl” in relation with a moiety of the structure:

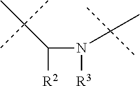

means that —R³ and —R² form the following structure:

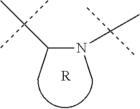

wherein R is a 3- to 10-membered heterocyclyl or an 8- to 11-membered heterobicyclyl.

Similarly, it is understood that the phrase “—R³ and —R¹ together with the nitrogen to which —R³ is attached, the carbon atom to which —R² is attached and the carbon atom to which —R¹ is attached form a 3- to 10-membered heterocyclyl or an 8- to 11-membered heterobicyclyl” in relation with a moiety of the structure:

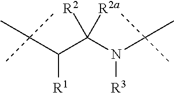

means that —R³ and —R¹ form the following structure:

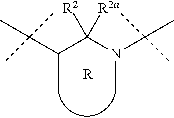

wherein R is a 3- to 10-membered heterocyclyl or an 8- to 11-membered heterobicyclyl.

As used herein, the term “excipient” refers to a diluent, adjuvant or vehicle with which the therapeutic, such as a drug or conjugate, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid) or can contain detergents, like Tween®, poloxamers, poloxamines, CHAPS, Igepal® or amino acids like, for example, glycine, lysine or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose and magnesium carbonate. Such compositions will contain a therapeutically effective amount of the drug or drug moiety, together with a suitable amount of excipient so as to provide the form for proper administration to the patient. The formulation should suit the mode of administration.

As used herein, the term “free form” of a drug refers to the drug in its unmodified, pharmacologically fully active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively charged ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine, guanine, thymine, cytosine or uracil analogues, may also be a type of small molecule drugs.

As used herein, the term “medium molecule drug” refers to drugs that are organic compounds which are not peptides and which are not proteins, and have a molecular weight ranging from and including 1 kDa to 7.5 kDa.

As used herein, the term “oligonucleotide” refers to double- or single-stranded RNA and DNA with preferably 2 to 1000 nucleotides and any modifications thereof. Modifications include for example, those which provide other chemical groups that incorporate additional charge, polarizability, hydrogen bonding, electrostatic interaction, and fluxionality to the nucleic acid ligand bases or to the nucleic acid ligand as a whole. Such modifications include for example, 20 to 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, unusual base-pairing combinations such as the isobases isocytidine and isoguanidine. Modifications can also include 3′ and 5′ modifications such as capping and change of stereochemistry. The term also includes aptamers.

As used herein, the term “peptide nucleic acids” refers to organic polymers having a peptidic backbone, i.e. a backbone in which the monomers are connected to each other through peptide linkages, to which nucleobases such as adenine, cytosine, guanine, thymine and uracil, are attached. In certain embodiments, the peptide backbone comprises N-(2-aminoethyl)-glycine.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” or “polymeric moiety” refers to a reagent or a moiety comprising one or more polymers or polymer moieties. A polymeric reagent or moiety may optionally also comprise one or more other moiety/moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

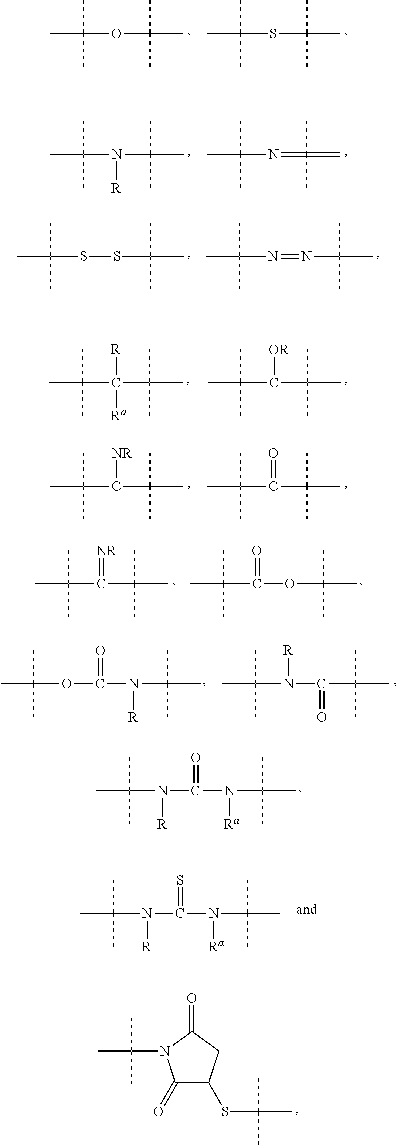

wherein
dashed lines indicate attachment to the remainder of the moiety or reagent;
—R and —Ra are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean number of monomers lies. An integer for “x” given as “about x” means that the arithmetic mean number of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments, such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

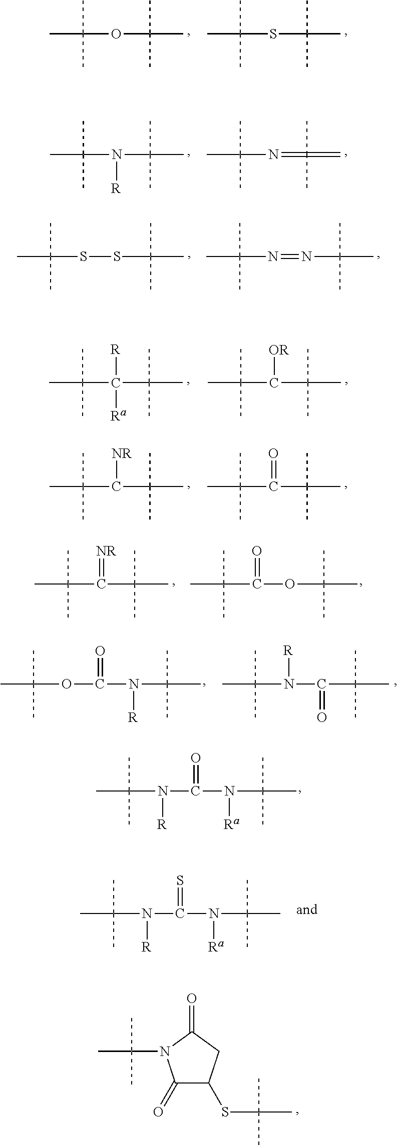

wherein
dashed lines indicate attachment to the remainder of the moiety or reagent;
—R and —Ra are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent. In certain embodiments, all ethylene glycol units of said moiety or reagent are present in one block; the remaining weight percentage of the PEG-based moiety or reagent are other moieties in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

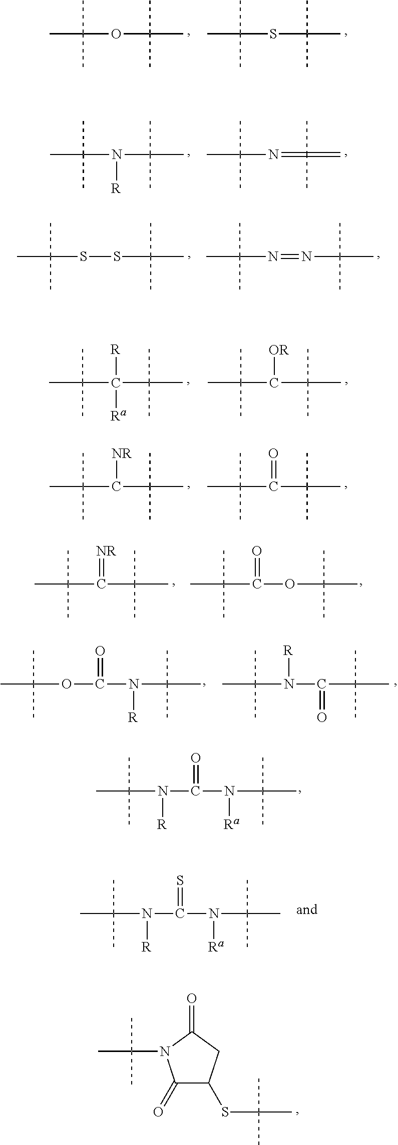

wherein
dashed lines indicate attachment to the remainder of the moiety or reagent, and
wherein —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hyaluronic acid-based” in relation to a moiety or reagent means that said moiety or reagent comprises hyaluronic acid. Such hyaluronic acid-based moiety or reagent comprises at least 10% (w/w) hyaluronic acid, such as at least 20% (w/w) hyaluronic acid, such as at least 30% (w/w) hyaluronic acid, such as at least 40% (w/w) hyaluronic acid, such as at least 50% (w/w) hyaluronic acid, such as at least 60% (w/w) hyaluronic acid, such as at least 70% (w/w) hyaluronic acid, such as at least 80% (w/w) hyaluronic acid, such as at least 90% (w/w) hyaluronic acid, or such as at least 95% (w/w) hyaluronic acid. The remaining weight percentage of the hyaluronic acid-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

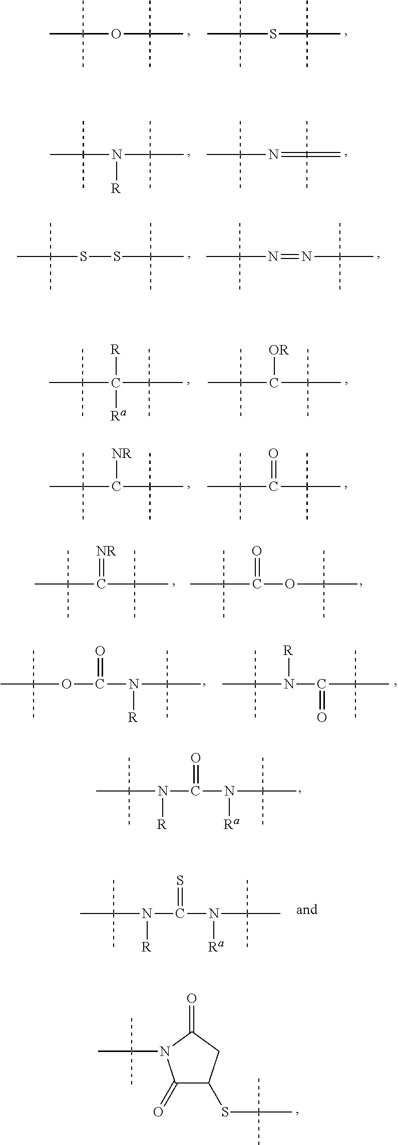

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 3- to 10-membered heterocyclyl, phenyl and naphthyl and may optionally be substituted.

As used herein, the term “substituted” means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
—R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR⁰³)(R^{x3a})—, —N(R^{x3})C(O)N(R^{x3a})—, and —OC(O)N(R^{x3})—;
each —R^{x1}, —R^{x1a}, —R^{x1b}, —R^{x2}, —R^{x3}, —R^{x3}a is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different.

In certain embodiments, a maximum of 6-H atoms of an optionally substituted molecule are independently replaced by a substituent, e.g. 5-H atoms are independently replaced by a substituent, 4-H atoms are independently replaced by a substituent, 3-H atoms are independently replaced by a substituent, 2-H atoms are independently replaced by a substituent, or 1-H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

In general, the term “comprise(s)” or “comprising” also encompasses “consist of” or “consisting of”.

In certain embodiments, all moieties -D of the conjugate are identical, i.e. have the same chemical structure. In such case all moieties -D of the conjugate derive from the same type of drug molecule. It is understood that this means that all moieties -D originate from the same parent drug, but that there may be molecular rearrangements that for example lead to the formation of different tautomeric forms.

In certain embodiments, the conjugate of the present invention comprises different moieties -D, i.e. comprises moieties -D with different chemical structures. These different structures derive from different types of drug molecules. In certain embodiments, the conjugate of the present invention comprises two different types of moieties -D. In certain embodiments, the conjugate of the present invention comprises three different types of moieties -D. In certain embodiments, the conjugate of the present invention comprises four different types of moieties -D. In certain embodiments, the conjugate of the present invention comprises five different types of moieties -D.

If the conjugates of the present invention comprise more than one type of -D, all moieties -D may be conjugated to the same type of -L¹- or may be conjugated to different types of -L¹-, i.e. a first type of -D may be conjugated to a first type of -L¹-, a second type of -D may be conjugated to a second type of -L¹- and so on. Using different types of -L¹- may, in certain embodiments, allow different release kinetics for different types of -D, such as for example a faster release for a first type of -D, a medium release for a second type of -D and a slow release for a third type of -D. Accordingly, in certain embodiments the conjugates of the present invention comprise one type of -L¹-. In certain embodiments, the conjugates of the present invention comprise two types of -L¹-. In certain embodiments, the conjugates of the present invention comprise three types of -L¹-. In certain embodiments, the conjugates of the present invention comprise four types of -L¹-.

In certain embodiments, the conjugates of the present invention comprise one type of -D and one type of -L¹-. In certain embodiments, the conjugates of the present invention comprise two types of -D and two types of -L¹-. In certain embodiments, the conjugates of the present invention comprise three types of -D and three types of -L¹-. In certain embodiments, the conjugates of the present invention comprise four types of -D and four types of -L¹-.

In certain embodiments, all moieties -L¹- of the conjugate have the same structure. In certain embodiments, the conjugate comprises two or more different types of moiety -L¹-, such as for example two, three, four or five different types of moiety -L¹-. Such two or more different types of moiety -L¹- may be conjugated to the same or different type of -D. Using different types of -L¹- allows releasing the same or different type of drug D-H from the conjugate of the present invention with different release half-lives, such as when combining a first group of moieties -L¹- with a short release half-life with a second group of moieties -L¹- with a long release half-life.

In certain embodiments, -D is selected from the group consisting of small molecule, medium size molecule, oligonucleotide, peptide nucleic acid, peptide and protein drug moieties.

In certain embodiments, -D is a small molecule drug moiety. In certain embodiments, such small molecule drug moiety is a nucleobase-based drug moiety.

In certain embodiments, -D is a peptide drug moiety.

In certain embodiments, -D is a protein drug moiety. In certain embodiments, such protein moiety is a monoclonal or polyclonal antibody or fragment or fusion thereof.

In certain embodiments, -D is a medium size molecule drug moiety. In certain embodiments, -D is an oligonucleotide drug moiety. In certain embodiments, -D is a peptide nucleic acid drug moiety.

In certain embodiments, -D is a nitrogen comprising drug moiety such as a primary or secondary amine-comprising drug moiety. In certain embodiments, -D is a hydroxyl-comprising drug moiety. In certain embodiments, -D is a carboxylic acid comprising drug moiety.

In certain embodiments, -D is a TNF inhibitor or a derivative thereof.

As used herein, the term “TNF inhibitor” refers to antibodies or fusion proteins that bind to TNF alpha.

In certain embodiments, -D is a TNF inhibitor selected from the group consisting of etanercept (Enbrel®), infliximab (Remicade®), adalimumab (Humira®), certolizumab pegol (Cimzia®) and golimumab (Simponi®).

In certain embodiments, -L¹- is a traceless linker, i.e. a linker that releases the drug in an unmodified form. In certain embodiments, -L¹- cleaves such that part of -L¹- remains attached to the released drug. The latter case may occur if -D is a carboxylic acid comprising drug which is attached via said carboxylic acid.

In certain embodiments, p is 0. In certain embodiments, p is 1.

In certain embodiments, -T- is

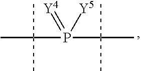

wherein ═Y⁴ and —Y⁵ are as defined in formula (I). In certain embodiments, -T- is

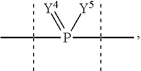

═Y⁴ is ═O and —Y⁵ is —O(R⁵), wherein —R⁵ is as defined in formula (I). In certain embodiments, -T- is

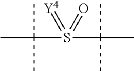

and ═Y⁴ is as defined in formula (I). In certain embodiments, -T- is

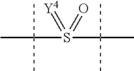

### and ═Y⁴ is ═O.

In certain embodiments, ═Y¹ and ═Y⁴ are independently selected from the group consisting of ═O and ═S.

In certain embodiments, ═Y¹ is ═O. In certain embodiments, ═Y¹ is ═S. In certain embodiments, ═Y¹ is ═N(R⁴), wherein —R⁴ is as defined in formula (I).

In certain embodiments, ═Y⁴ is ═O. In certain embodiments, ═Y⁴ is ═S. In certain embodiments, ═Y⁴ is ═N(R⁴), wherein —R⁴ is as defined in formula (I).

In certain embodiments, —Y²— is —O—. In certain embodiments, —Y²— is —S—. In certain embodiments, —Y²— is —N(R⁴)—, wherein —R⁴ is as defined in formula (I).

In certain embodiments, —Y³— is —O—. In certain embodiments, —Y³— is —S—. In certain embodiments, —Y³— is —N(R⁴)—, wherein —R⁴ is as defined in formula (I).

In certain embodiments, —Y⁶— is —O—. In certain embodiments, —Y⁶— is —S—. In certain embodiments, —Y⁶— is —N(R⁴)—, wherein —R⁴ is as defined in formula (I).

In certain embodiments, —Y⁵— is —O(R⁵). In certain embodiments, —Y⁵— is —S(R⁵). In certain embodiments, —Y⁵— is —N(R⁴)(R^{4a}).

In certain embodiments, —X— is C₂₋₃ alkyl. In certain embodiments, —X— is selected from the group consisting of ethyl, propyl and isopropyl, which ethyl, propyl and isopropyl are optionally substituted by a halogen. In certain embodiments, —X— is ethyl. In certain embodiments, —X— is propyl. In certain embodiments, —X— is isopropyl.

In certain embodiments, —X— is of formula (a) and —R*— is selected from the group consisting of C₃₋₁₀ cycloalkyl and 3- to 10-membered heterocyclyl. In certain embodiments, —X— is of formula (a) and —R*— is C₃₋₁₀ cycloalkyl.

In certain embodiments, —X— of formula (a) has the following structure:

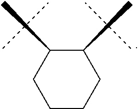

In certain embodiments, —X— is of formula (b) and —R*— is selected from the group consisting of C₃₋₁₀ cycloalkyl and 3- to 10-membered heterocyclyl. In certain embodiments, —X— is of formula (b) and —R*— is C₃₋₁₀ cycloalkyl.

In certain embodiments, —X— of formula (b) has the following structure:

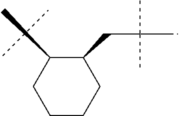

In certain embodiments, —X— is of formula (c).

In certain embodiments, t is 0. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7. In certain embodiments, t is 8. In certain embodiments, t is 9. In certain embodiments, t is 10.

In certain embodiments, -A- is a ring selected from the group consisting of monocyclic or bicyclic aryl and heteroaryl and t is selected from the group consisting of 0, 1, 2, 3, 4, 5 and 6. In certain embodiments, -A- is a monocyclic aryl or heteroaryl ring and t is selected from the group consisting of 0, 1, 2, 3 and 4.

In certain embodiments, -A- is selected from the group consisting of:

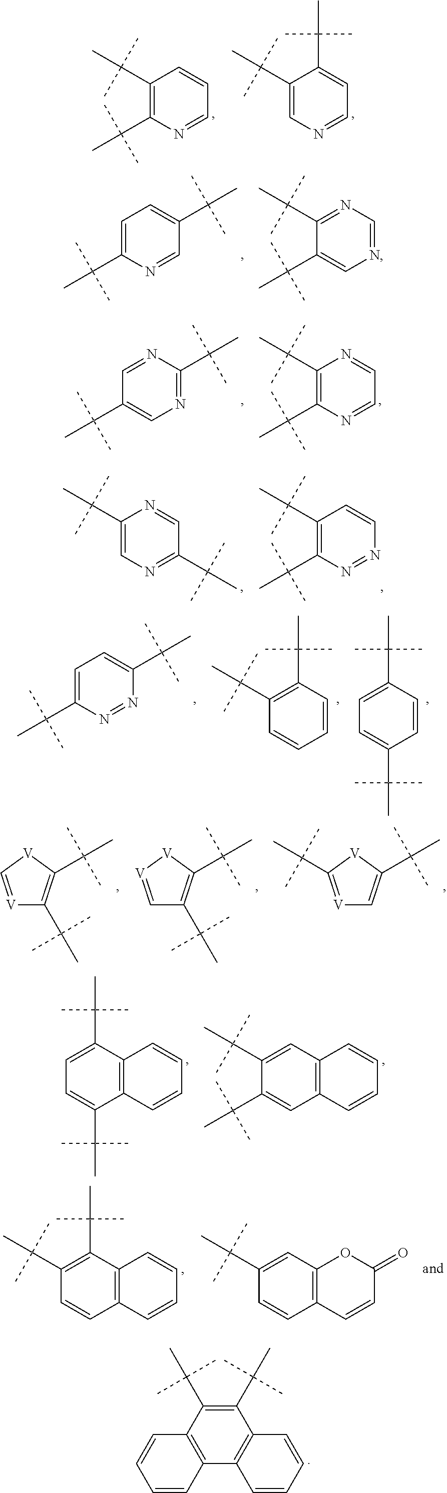

wherein each V is independently selected from the group consisting of O, S and N.

In certain embodiments, -M- is of formula (i). In certain embodiments, -M- is of formula (ii).

In certain embodiments, —Y⁷— is —O—. In certain embodiments, —Y⁷— is —S—. In certain embodiments, —Y⁷— is —N(R⁴)—, wherein —R⁴ is as defined in formula (I).

In certain embodiments, ═Y⁸ is ═O. In certain embodiments, ═Y⁸ is ═S. In certain embodiments, ═Y⁸ is ═N(R⁴), wherein —R⁴ is as defined in formula (I).

In certain embodiments, n is 1. In certain embodiments, n is 2. In certain embodiments, n is 3.

In certain embodiments, —R¹, —R^{1a}, —R², —R^{2a} are independently selected from the group consisting of —H, —C(O)OH, halogen, —NO₂, —CN, —OH, -T*, C₁₋₆ alkyl, C₂₋₆ alkenyl and C₂₋₆ alkynyl. In certain embodiments, —R¹, —R^{1a}, —R², —R^{2a} are independently selected from the group consisting of —H, C₁₋₆ alkyl, C₂₋₆ alkenyl and C₂₋₆ alkynyl.

In certain embodiments, —R¹ is —H. In certain embodiments, —R¹ is —C(O)OH. In certain embodiments, —R¹ is halogen. In certain embodiments, —R¹ is —NO₂. In certain embodiments, —R¹ is —CN. In certain embodiments, —R¹ is —OH. In certain embodiments, —R¹ is -T*. In certain embodiments, —R¹ is C₁₋₆ alkyl. In certain embodiments, —R¹ is C₂₋₆ alkenyl. In certain embodiments, —R¹ is C₂₋₆ alkynyl.

In certain embodiments, —R^{1a} is —H. In certain embodiments, —R^{1a} is —C(O)OH. In certain embodiments, —R^{1a} is halogen. In certain embodiments, —R^{1a} is —NO₂. In certain embodiments, —R^{1a} is —CN. In certain embodiments, —R^{1a} is —OH. In certain embodiments, —R^{1a} is -T*. In certain embodiments, —R^{1a} is C₁₋₆ alkyl. In certain embodiments, —R^{1a} is C₂₋₆ alkenyl. In certain embodiments, —R^{1a} is C₂₋₆ alkynyl.

In certain embodiments, —R² is —H. In certain embodiments, —R² is —C(O)OH. In certain embodiments, —R² is halogen. In certain embodiments, —R² is —NO₂. In certain embodiments, —R² is —CN. In certain embodiments, —R² is —OH. In certain embodiments, —R² is -T*. In certain embodiments, —R² is C₁₋₆ alkyl. In certain embodiments, —R² is C₂₋₆ alkenyl. In certain embodiments, —R² is C₂₋₆ alkynyl.

In certain embodiments, —R^{2a} is —H. In certain embodiments, —R^{2a} is —C(O)OH. In certain embodiments, —R^{2a} is halogen. In certain embodiments, —R^{2a} is —NO₂. In certain embodiments, —R^{2a} is —CN. In certain embodiments, —R^{2a} is —OH. In certain embodiments, —R^{2a} is -T*. In certain embodiments, —R^{2a} is C₁₋₆ alkyl. In certain embodiments, —R^{2a} is C₂₋₆ alkenyl. In certain embodiments, —R^{2a} is C₂₋₆ alkynyl.

In certain embodiments, —R⁵, each —R⁶, each —R^{6a}, each —R⁷ and each —R^{7a} are independently selected from the group consisting of —H, -T*, C₁₋₆ alkyl, C₂₋₆ alkenyl and C₂₋₆ alkynyl. In certain embodiments, —R⁵, each —R⁶, each —R^{6a}, each —R⁷ and each —R^{7a} are independently selected from the group consisting of —H, C₁₋₆ alkyl, C₂₋₆ alkenyl and C₂₋₆ alkynyl.

In certain embodiments, —R⁵ is —H. In certain embodiments, —R⁵ is -T*. In certain embodiments, —R⁵ is C₁₋₆ alkyl. In certain embodiments, —R⁵ is C₂₋₆ alkenyl. In certain embodiments, —R⁵ is C₂-6 alkynyl.

In certain embodiments, —R⁶ is —H. In certain embodiments, —R⁶ is -T*. In certain embodiments, —R⁶ is C₁₋₆ alkyl. In certain embodiments, —R⁶ is C₂₋₆ alkenyl. In certain embodiments, —R⁶ is C₂₋₆ alkynyl.

In certain embodiments, —R^{6a} is —H. In certain embodiments, —R^{6a} is -T*. In certain embodiments, —R^{6a} is C₁₋₆ alkyl. In certain embodiments, —R^{6a} is C₂₋₆ alkenyl. In certain embodiments, —R^{6a} is C₂₋₆ alkynyl.

In certain embodiments, —R⁷ is —H. In certain embodiments, —R⁷ is -T*. In certain embodiments, —R⁷ is C₁₋₆ alkyl. In certain embodiments, —R⁷ is C₂₋₆ alkenyl. In certain embodiments, —R⁷ is C₂₋₆ alkynyl.

In certain embodiments, —R^{7a} is —H. In certain embodiments, —R^{7a} is -T*. In certain embodiments, —R^{7a} is C₁₋₆ alkyl. In certain embodiments, —R^{7a} is C₂₋₆ alkenyl. In certain embodiments, —R^{7a} is C₂₋₆ alkynyl.

In certain embodiments, -T* is phenyl. In certain embodiments, -T* is naphtyl. In certain embodiments, -T* is indenyl. In certain embodiments, -T* is indanyl. In certain embodiments, -T* is tetralinyl. In certain embodiments, -T* is C₃₋₁₀ cycloalkyl. In certain embodiments, -T* is 3- to 10-membered heterocyclyl. In certain embodiments, -T* is 8- to 11-membered heterobicyclyl. In certain embodiments, -T* is substituted with one or more —R^{x}. In certain embodiments, -T* is not substituted with —R^{x}.

In certain embodiments, —R^{x} is selected from the group consisting of —H, —NO₂, —OCH₃, —CN, —N(R⁴)(R^{4a}), —OH, —C(O)OH and C₁₋₆ alkyl. In certain embodiments, —R^{x} is —H. In certain embodiments, —R^{x} is —NO₂. In certain embodiments, —R^{x} is —OCH₃. In certain embodiments, —R^{x} is —CN. In certain embodiments, —R^{x} is —N(R⁴)(R^{4a}). In certain embodiments, —R^{x} is —OH. In certain embodiments, —R^{x} is —C(O)OH. In certain embodiments, —R^{x} is C₁₋₆ alkyl.

In certain embodiments, —R³, —R⁴ and —R^{4a} are independently selected from the group consisting of —H and C₁₋₆ alkyl.

In certain embodiments, —R³ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁴ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In certain embodiments, —R⁴ is —H.

In certain embodiments, —R^{4a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In certain embodiments, —R^{4a} is —H.

In certain embodiments, —R³ and —R¹ together with the nitrogen to which —R³ is attached, the carbon atom to which —R² is attached and the carbon atom to which —R¹ is attached form a 4- to 10-membered heterocyclyl or an 8- to 11-membered heterobicyclyl, which 4- to 10-membered heterocyclyl or 8- to 11-membered heterobicyclyl is optionally substituted with one or more —R^{x} which are the same or different. In certain embodiments, —R³ and —R¹ together with the nitrogen to which —R³ is attached, the carbon atom to which —R² is attached and the carbon atom to which —R¹ is attached form a 4- to 10-membered heterocyclyl which 4- to 10-membered heterocyclyl is optionally substituted with one or more —R^{x} which are the same or different.

In certain embodiments, —R¹ and —R² are joined together to form a carbohydrate-nucleobase system of formula (ai) to (di):

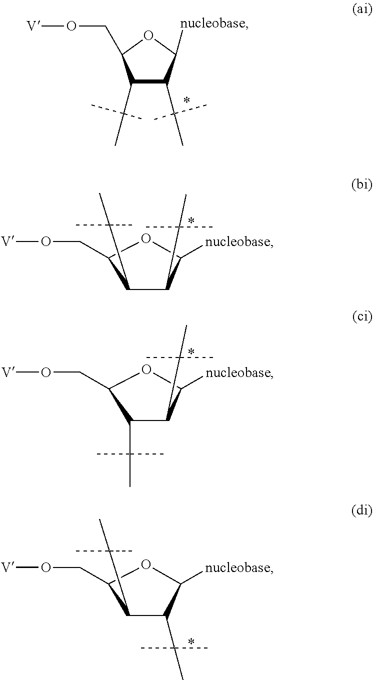

wherein the dashed line marked with an asterisk indicates the attachment to —W— of formula (I) and the unmarked dashed line indicates the attachment to —Y⁶— of formula (I) or vice versa; and
—V′ is selected from the group consisting of —H and protecting group.

In certain embodiments, the nucleobase of formula (ai), (bi), (ci) or (di) which is attached to the position 1′ of the ring is selected from the group consisting of adenine, cytosine, guanine, thymine, uracil and other natural and non-natural nucleobases.

In certain embodiments, the nucleobase of formula (ai), (bi), (ci) or (di) is selected from the group consisting of adenine, cytosine, guanine, thymine, uracil, 5-methylcytosine, pseudouridine, dihydrouridine, inosine, 7-methylguanosine, hypoxanthine and xanthine. In certain embodiments, the nucleobase of formula (ai), (bi), (ci) or (di) is selected from the group consisting of adenine, cytosine, guanine, thymine and uracil. In certain embodiments, the nucleobase of formula (ai), (bi), (ci) or (di) is adenine. In certain embodiments, the nucleobase of formula (ai), (bi), (ci) or (di) is cytosine. In certain embodiments, the nucleobase of formula (ai), (bi), (ci) or (di) is guanine. In certain embodiments, the nucleobase of formula (ai), (bi), (ci) or (di) is thymine. In certain embodiments, the nucleobase of formula (ai), (bi), (ci) or (di) is uracil. In certain embodiments, the nucleobase of formula (ai), (bi), (ci) or (di) is 5-methylcytosine. In certain embodiments, the nucleobase of formula (ai), (bi), (ci) or (di) is pseudouridine. In certain embodiments, the nucleobase of formula (ai), (bi), (ci) or (di) is dihydrouridine. In certain embodiments, the nucleobase of formula (ai), (bi), (ci) or (di) is inosine. In certain embodiments, the nucleobase of formula (ai), (bi), (ci) or (di) is 7-methylguanosine. In certain embodiments, the nucleobase of formula (ai), (bi), (ci) or (di) is hypoxanthine. In certain embodiments, the nucleobase of formula (ai), (bi), (ci) or (di) is xanthine.

In certain embodiments, the nucleobase of formula (ai), (bi), (ci) or (di) comprises a fluorescent group.

In certain embodiments, —R¹ and —R² are joined together to form a carbohydrate-nucleobase system of formula (ai). In certain embodiments, —R¹ and —R² are joined together to form a carbohydrate-nucleobase system of formula (bi). In certain embodiments, —R¹ and —R² are joined together to form a carbohydrate-nucleobase system of formula (ci). In certain embodiments, —R¹ and —R² are joined together to form a carbohydrate-nucleobase system of formula (di).

In certain embodiments, —R¹ and —R² are joined together to form a carbohydrate-nucleobase system such as a ribose ring system of formula (ei) to (ji):

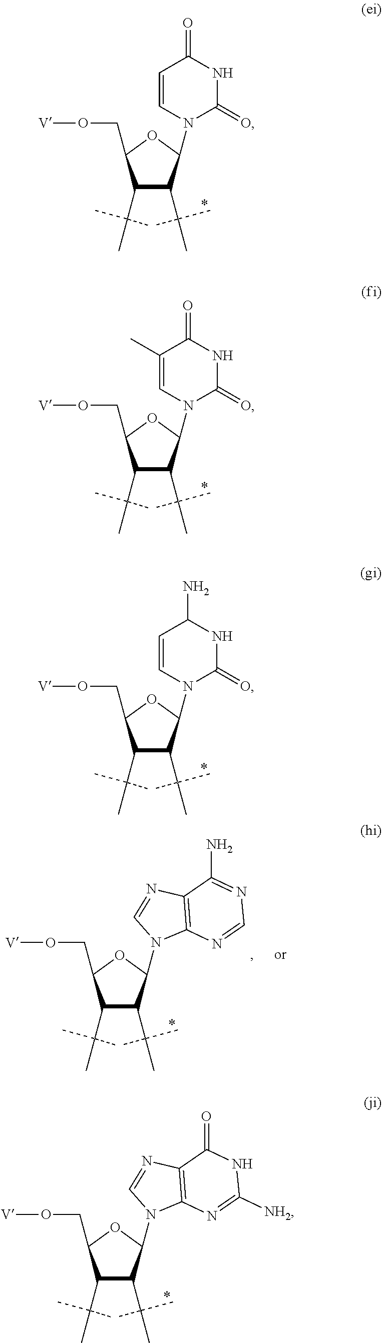

wherein the dashed line marked with an asterisk indicates the attachment to —W— of formula (I) and the unmarked dashed line indicates the attachment to —Y⁶— of formula (I) or vice versa; and
—V′ is selected from the group consisting of —H and a protecting group.

In certain embodiments, —V′ is —H. In certain embodiments, —V′ is a protecting group.

In certain embodiments, the protecting group is an acyl group such as an acyl group selected from the group consisting of formyl, acetyl, chloroacetyl, dichloroacetyl, trichloroacetyl, cyanoacetyl, mono-methyl malonate, mono-ethyl malonate, methoxyacetyl, ethoxyacetyl, t-butoxyacetyl, phenoxyacetyl, t-butylphenoxyacetyl, glycolate, acetylglycolate, propionate, 2-chloropropionate, 3-chloropropionate, 2-cyanopropionate, 3-cyanopropionate, N-acetyl-glycinate, N-trifluoroacetyl glycinate, N-acetyl alanylate, N-acetyl phenylalanylate, N-trifluoroacetyl phenylalanylate, N-acetyl valinylate, N-trifluoroacetyl valinylate, N-acetyl valinyl-citrunyllate, butyrate, isobutyrate, pivaloyl, levulinate, monomethyl oxalate, mono-ethyloxalate, mono-methyl succinate, mono-ethyl succinate, hydroxyl butyrate, acetoxybutyrate, acetylbutyrate, hexanoate, palmitate, stearate, benzoate, chloro-benzoate, dichloro-benzoate, pentachlorobenzoate, cyano-benzoate, aminobenzoate, acetamino-benzoate, mono-methyl phthalate, mono-ethyl phthalate, methoxy-benzoate, trimethoxybenzoate, trifluoromethylbenzoate, dimethylaminobenzoate and methylsulfonylbenzoate. In certain embodiments, —V′ is an acyl group such as an acyl group selected from the group consisting of formyl, acetyl, propionyl, acrylyl, pivaloyl and benzoyl.

In certain embodiments, —V′ is pivaloyl or benzoyl. In certain embodiments, —V′ and -E are the same such as for example —V′ and -E are each an acyl group. In certain embodiments, —V′ is an acyl group and -E is a cleavable moiety other than an acyl group. In certain embodiments, an acyl group is hydrolyzable in the presence of any one of the numerous hydrolase enzymes existing in vivo.

In certain embodiments, —V′ is

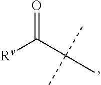

wherein —R is C₁₋₆ alkyl, and wherein C₁₋₆ alkyl is optionally substituted by a halogen.

In certain embodiments, —V′ is a hydroxyl protecting group such as methoxymethyl ether (MOM), benzylmethyl ether (BOM), benzyl ether, p-methoxybenzyl ether (PMB), trityl ether, silyl ether (TMS, TIPS), or any of the hydroxyl protecting groups described in the art. In certain embodiments, —V′ is an alcohol protecting group selected from the group consisting of t-butyldimethylsilyl, diethylisopropylsilyl, triphenylsilyl, formate, methoxymethylcarbonate, t-butylcarbonate, 9-fluorenylmethylcarbonate, N-phenylcarbamate, 4,4′-dimethoxytrityl, monomethoxytrityl, trityl and pixyl.

In certain embodiments, -E is of formula (a′):

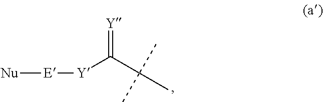

wherein the dashed line indicates the attachment to —W— of formula (I);
-Nu is a nucleophile;
-E′- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R^{x}, which are the same or different;
   -Q- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -Q- is independently optionally substituted with one or more —R^{x}, which are the same or different;
—Y′— is selected from the group consisting of —O—, —C(R⁶)(R^{6a})—, —N(R⁴)— and —S—;
═Y″ is selected from the group consisting of ═O, ═S and ═N(R⁴); and
—R^{x}, —R⁴, —R⁶ and —R^{6a} are used as defined in formula (I).

In certain embodiments, -Nu of formula (a′) is selected from the group consisting of primary, secondary or tertiary amine and amide. In certain embodiments, -Nu of formula (a′) is a primary amine. In certain embodiments, -Nu of formula (a′) is a secondary amine. In certain embodiments, -Nu of formula (a′) is a tertiary amine. In certain embodiments, -Nu of formula (a′) is an amide.

In certain embodiments, -E′- of formula (a′) is selected from the group consisting of C₁₋₆ alkyl, C₂₋₆ alkenyl and C₂₋₆ alkynyl.

In certain embodiments, —Y′— of formula (a′) is —O—. In certain embodiments, —Y′— of formula (a′) is —C(R⁶)(R^{6a})—. In certain embodiments, —Y′— of formula (a′) is —N(R⁴)—. In certain embodiments, —Y′— of formula (a′) is —S—.

In certain embodiments, ═Y″ of formula (a′) is ═O. In certain embodiments, ═Y″ of formula (a′) is ═S. In certain embodiments, ═Y″ of formula (a′) is ═N(R⁴).

In certain embodiments, -E is of formula:

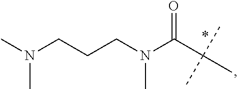

wherein the dashed line marked with the asterisk indicates the attachment to —W— of formula (I).

In certain embodiments, -E is of formula:

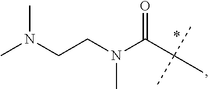

wherein the dashed line marked with an asterisk indicates the attachment to —W— of formula (I).

In certain embodiments, -E is attached to —W— via a moiety of formula:

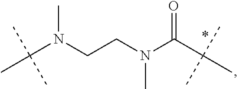

wherein the dashed line marked with an asterisk indicates the attachment to -E of formula (I) and the dashed line marked with an asterisk indicates the attachment to —W— of formula (I).

In certain embodiments, —W— of formula (I) is —N(R³)— and -E of formula (I) is as described for -L¹ of formula (I) disclosed in WO2009/095479A2. In certain embodiments, —W— of formula (I) is —N(R³)— and -E of formula (I) is as described for -L¹ of formula (I) disclosed in WO2011/089216A1. In certain embodiments, —W— of formula (I) is —N(R³)— and -E of formula (I) is as described for -L¹ of formula (I) disclosed in WO2016/196124A2. In certain embodiments, —W— of formula (I) is —O— or —N(R³)— and -E of formula (I) is as described for -L¹ of formula (I) disclosed in WO2016/020373A1. In certain embodiments, —W— of formula (I) is —O— and -E of formula (I) is as described for -L¹ of formula (I) disclosed in WO2013/160340A1. In certain embodiments, —W— of formula (I) is —N(R³)— and -E of formula (I) is as described for -L¹- of formula (IV) disclosed in WO 2017/148883A1.

In certain embodiments, -E is cleavable at physiological pH. In certain embodiments, -E is cleavable by hydrolysis at physiological pH. In certain embodiments, -E is cleavable by bases at physiological pH via the β-elimination or is cleavable in an autocatalytic manner that starts with deprotonation of the most basic amino group in the -E group.

In certain embodiments, -E is cleavable by bases at physiological pH, via the β-elimination mechanism (e.g. a β-eliminative trigger). In such cases, -E is selected from the group consisting of the following structures:

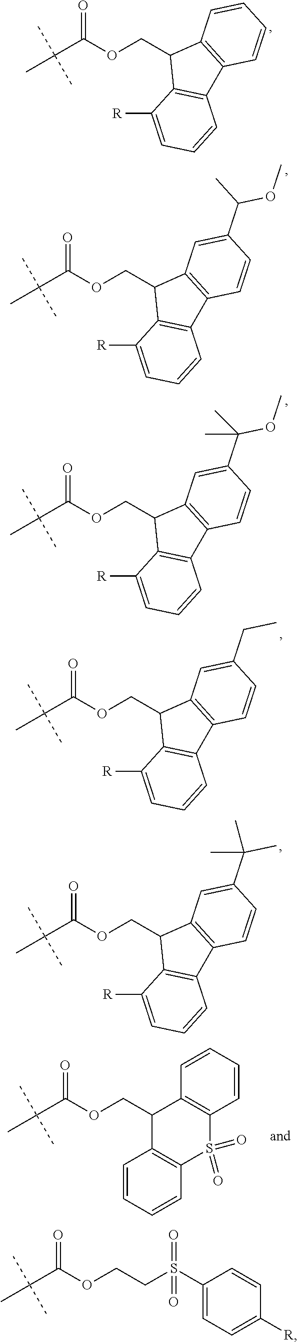

wherein —R is selected from the group consisting of —H, C₁₋₁₀ alkyl, —OH, —NO₂, —CN, halogen and acetyl.

In such cases, induction of electron-withdrawing substituents —R can increase the rate of β-elimination and liberation of free —WH.

In certain embodiments, -E has the following structure:

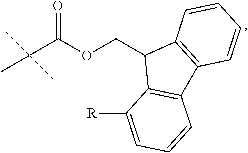

wherein —R is selected from the group consisting of —H, C₁₋₁₀ alkyl, —OH, —NO₂, —CN, halogen and acetyl.

In certain embodiments, -E has the following structure which comprises the electron withdrawing —SO₂— group:

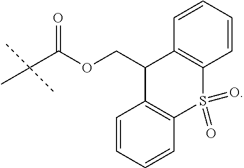

In certain embodiments, -E has the following structure:

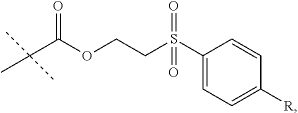

wherein —R is selected from the group consisting of —H, C₁₋₁₀ alkyl, —OH, —NO₂, —CN, halogen and acetyl. These functional groups cleave via a β-elimination mechanism at about pH 7.4 and the rate of this process can be controlled by —R.

In certain embodiments, -E has the following structure:

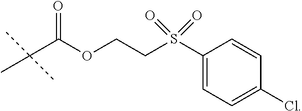

In certain embodiments, -E is an acyl group selected from the group consisting of formyl, acetyl, chloroacetyl, acryloate, dichloroacetyl, trichloroacetyl, cyanoacetyl, mono-methyl malonate, mono-ethyl malonate, methoxyacetyl, ethoxyacetyl, t-butoxyacetyl, phenoxyacetyl, t-butylphenoxyacetyl, glycolate, acetylglycolate, propionate, 2-chloropropionate, 3-chloropropionate, 2-cyanopropionate, 3-cyanopropionate, N-acetyl-glycinate, N-trifluoroacetyl glycinate, N-acetyl alanylate, N-acetyl phenylalanylate, N-trifluoroacetyl phenylalanylate, N-acetyl valinylate, N-trifluoroacetyl valinylate, N-acetyl valinyl-citrunyllate, butyrate, isobutyrate, pivaloyl, levulinate, monomethyl oxalate, mono-ethyloxalate, mono-methyl succinate, mono-ethyl succinate, hydroxyl butyrate, acetoxybutyrate, acetylbutyrate, hexanoate, palmitate, stearate, benzoate, chloro-benzoate, dichloro-benzoate, pentachlorobenzoate, cyano-benzoate, aminobenzoate, acetamino-benzoate, mono-methyl phthalate, mono-ethyl phthalate, methoxy-benzoate, trimethoxybenzoate, trifluoromethylbenzoate, dimethylaminobenzoate and methylsulfonylbenzoate.

In certain embodiments, -E is an acyl group selected from the group consisting of formyl, acetyl, propionate, acryloate, pivalyol and benzoate. In certain embodiments, -E is an acyl group such as pivaloyl or benzoate.

In certain embodiments, -E is selected from the group consisting of acetal, ortho-ester and substituted triphenylmethylethers.

In certain embodiments, -E is selected from the group consisting of tetrahydrofuranyl, 4-methoxytetrahydropyran-4-yl, 1,5-dicarbo-methoxypentanyl, methoxy isopropyl acetal, methoxy cyclohexenyl acetal, dimethoxytrityl, trimethoxytrityl and pixyl.

In certain embodiments, -E is an acyl group selected from the group consisting of formyl, acetyl, propionyl, acrylyl, pivaloyl and benzoyl. In certain embodiments, -E is a group that may be cleavable by an esterase such as an acyl group, carbonate ester or O-methyl-acyl ester.

In certain embodiments, —W— is —N(R³)— and -E is a cleavable moiety of formula:

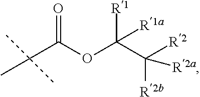

wherein —R′¹ and —R′^{1a} are independently selected from the group consisting of —H, C₃₋₁₀ cycloalkyl and C₁₋₆ alkyl, wherein C₁₋₆ alkyl is optionally substituted with one or more —R′³, which are the same or different; and wherein C₁₋₆ alkyl, C₂₋₆ alkenyl and C₂₋₆ alkynyl are optionally interrupted by one or more groups selected from the group consisting of -T″-, —C(O)O—, —O—, —C(O)—, —C(O)N(R′⁴)—, —S(O)₂N(R′⁴)—, —S(O)N(R′⁴)—, —S(O)₂—, —S(O)—, —N(R′⁴)S(O)₂N(R′^{4a})—, —S—, —N(R′⁴)—, —OC(OR′⁴)(R′^{4a})—, —N(R′⁴)C(O)N(R′^{4a})— and —OC(O)N(R′⁴)—;
—R′², —R′^{2a}, —R′^{2b} are independently selected from the group consisting of —H, —CN, —NO₂, —C(O)R′⁴, —S(O)R′⁴, —S(O)₂R′⁴, -T″ and C₁₋₆ alkyl, wherein C₁₋₆ alkyl is optionally substituted with one or more —R′³, which are the same or different; and wherein C₁₋₆ alkyl, C₂₋₆ alkenyl and C₂₋₆ alkynyl are optionally interrupted by one or more groups selected from the group consisting of -T″-, —C(O)O—, —O—, —C(O)—, —C(O)N(R′⁴)—, —S(O)₂N(R′⁴)—, —S(O)N(R′⁴)—, —S(O)₂—, —S(O)—, —N(R′⁴)S(O)₂N(R′^{4a})—, —S—, —N(R′⁴)—, —OC(OR′⁴)(R′^{4a})—, —N(R′⁴)C(O)N(R′^{4a})— and —OC(O)N(R′⁴)—; and wherein each -T″- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T″- is independently optionally substituted with one or more —R′³, which are the same or different;
   wherein —R′³ is selected from the group consisting of —H, —NO₂, —OCH₃, —CN, —N(R′⁴)(R′^{4a}), —OH, —C(O)OH and C₁₋₆ alkyl; wherein C₁₋₆ alkyl is optionally substituted with one or more halogen, which are the same or different;
   wherein —R′⁴ and —R′^{4a} are selected from the group consisting of —H and C₁₋₆ alkyl; and wherein
optionally, —R′² and —R′^{2a} are joined together with the carbon atom to which they are attached to form C₃₋₁₀ cycloalkyl, 3- to 10-membered heterocyclyl, 8- to 11-membered heterobicyclyl, phenyl, naphthyl, indenyl, indanyl or tetralinyl.

In certain embodiments, -E is cleavable at a pH of about 1.5 to about 8.5. In certain embodiments, -E is cleavable at a pH of 1.5 to 8.5.

In certain embodiments, -E is cleavable at basic pH such as at a pH of about 7.8 to about 8.5. In certain embodiments, -E is cleavable at a pH of about 7.8. In certain embodiments, -E is cleavable at a pH of about 8. In certain embodiments, -E is cleavable at a pH of about 8.5. In certain embodiments, -E is cleavable at a pH of 7.8. In certain embodiments, -E is cleavable at a pH of 8. In certain embodiments, -E is cleavable at a pH of 8.5.

In certain embodiments, -E is cleavable at acidic pH such as at a pH of about 1.5 to about 6.3. In certain embodiments, -E is cleavable at a pH of about 1.5. In certain embodiments, -E is cleavable at a pH of about 3. In certain embodiments, -E is cleavable at a pH of about 4. In certain embodiments, -E is cleavable at a pH of about 5. In certain embodiments, -E is cleavable at a pH of about 6. In certain embodiments, -E is cleavable at a pH of about 6.3. In certain embodiments, -E is cleavable at a pH of 1.5 to 6.3. In certain embodiments, -E is cleavable at a pH of 1.5. In certain embodiments, -E is cleavable at a pH of 3. In certain embodiments, -E is cleavable at a pH of 4. In certain embodiments, -E is cleavable at a pH of 5. In certain embodiments, -E is cleavable at a pH of 6. In certain embodiments, -E is cleavable at a pH of 6.3.

In certain embodiments, -E is non-enzymatically cleavable at acidic pH such as at pH of about 1.5 to about 6.3 or at physiological pH. In certain embodiments, -E is non-enzymatically cleavable at physiological pH.

In certain embodiments, -E is non-enzymatically cleavable at acidic pH such as at pH of about 1.5 to about 6.3. Any acid-cleavable alcohol protecting group can be used as -E. In certain embodiments, -E is an acid-cleavable alcohol protecting group such as an acetal, ortho-ester or phenyl substituted ester. In such cases -E is selected from the group consisting of tetrahydrofuran, 4-methyltetrahydropyran, 4-methoxytetrahydropyran-4-yl, 3,4-methylenedioxy-N-methylphentermine, 1,5-dicarboxymethoxypentanyl, methoxy isopropyl acetal, methoxy cyclohexenyl acetal, dimethoxytrityl, trimethoxytrityl and pixyl group.

In certain embodiments, -E is selected from the group consisting of acyl group, O-methyl-acyl group, methyl azido group, sugar residue, protected acetal and carbonate ester.

In certain embodiments, -E is cleavable by an enzyme selected from the group consisting of esterases, specific or unspecific peptidases, reductases, oxidases, glycosidases, hydrolases, glycosyl transferases and transaminases. In certain embodiments, -E is cleavable by an enzyme selected from the group consisting of esterases, reductases, oxidases, glycosidases, hydrolases and glycosyl transferases.

In certain embodiments, -E is a cleavable moiety that upon cleavage liberates a free —WH group where H is hydrogen. In certain embodiments, -E is cleavable by one of the following enzymes:

a) Esterases

All esters, carbonates and methyloxy esters may be hydrolyzed by an enzyme such as an esterase. The reactivity of these functional groups in the enzymatic reaction can be modulated by selection of a carboxylic acid component of the ester functional group containing different electron donating groups or by making the ester sterically hindered. Both the acid component and the alcohol component of the ester may be sterically hindered.

b) Reductases

Methyl-dithioethers, methyl azido group and 2-oxymethyleneantraquinone carbonates (MAQC) are examples of cleavable moieties -E that both can be cleaved by a reductase enzyme. For example, a moiety -E that may be cleavable by a reductase is a methyl azido group, or a moiety of the following formula:

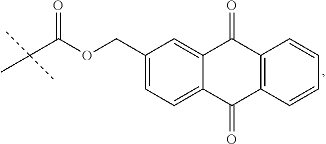

wherein the dashed line indicates the attachment to —W— of formula (I).

In certain embodiments, —W— is —O— and -E has the following structure:

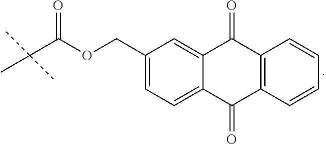

In certain embodiments, —W— is —NH— and -E has the following structure:

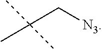

c) Glycosidases

If —WE is a heteroatom substituted by a sugar residue forming a glycosidic bond with the rest of the conjugate of formula (I), then a glycosidase can cleave -E in vivo and liberate the free —WH.

In certain embodiments, -E is cleavable by an enzyme such as glycoside hydrolase. In certain embodiments, -E is cleavable by an enzyme such as esterase. In certain embodiments, -E is a residue of a sugar selected from the group consisting of glucose, galactose, mannose and glucuronic acid. In certain embodiments, -E is selected from the group consisting of acyl group, carbonate ester and O-methyl-acyl ester.

In certain embodiments, -E is cleavable by a glycosidase. In such a case, -E is a sugar moiety such as a glucose, galactose or mannose moiety.

In certain embodiments, -E comprises a dithio group which is cleavable by a biogenic thiol. In certain embodiments, -E is cleavable by glutathione.

In certain embodiments, —W— is —O— and -E is selected from the group consisting of:

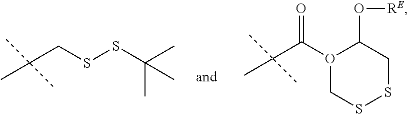

wherein the dashed line indicates the attachment to —W— and —R^{E} is selected from the group consisting of C₁₋₆ alkyl and benzyl.

In certain embodiments, —W— is —NH— and -E is selected from the group consisting of:

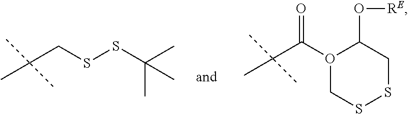

wherein the dashed line indicates the attachment to —W— and —R^{E} is selected from the group consisting of C₁₋₆ alkyl and benzyl.

In certain embodiments, —W— is —O— and -E is of formula:

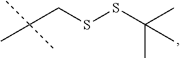

wherein the dashed line indicates the attachment to —W—.

In certain embodiments, —W— is —N(R³)— and -E is of formula:

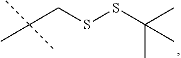

wherein the dashed line indicates the attachment to —W—.

In certain embodiments, -E is a cleavable moiety selected from the group consisting of:

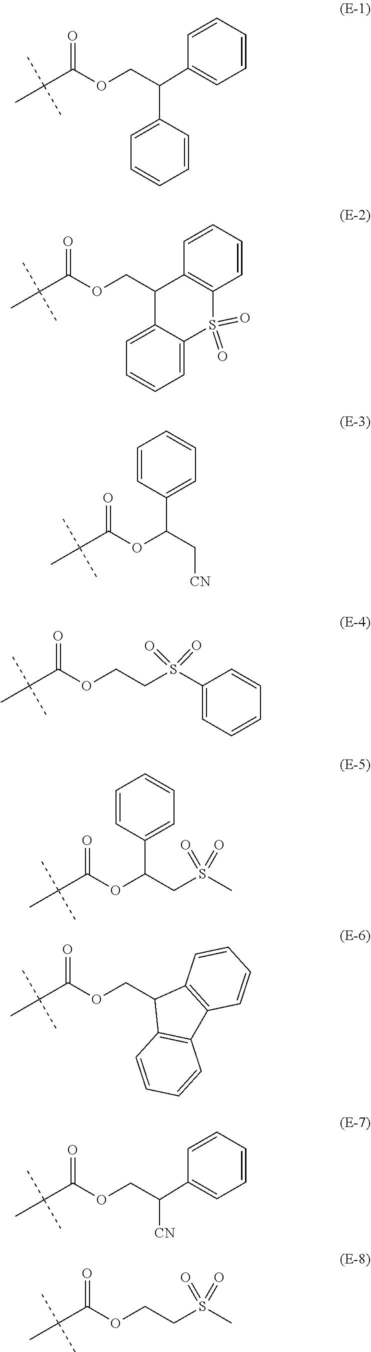

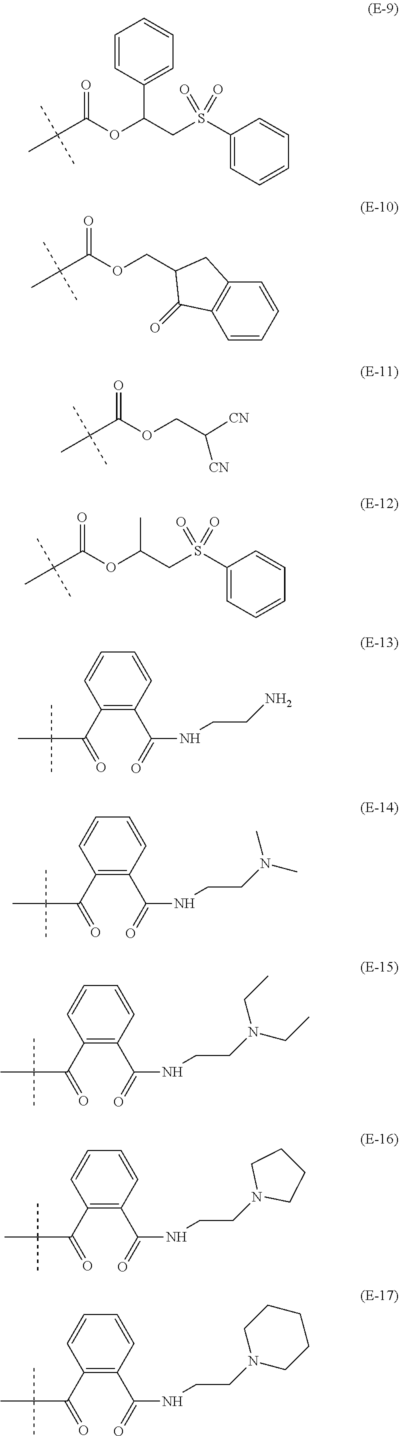

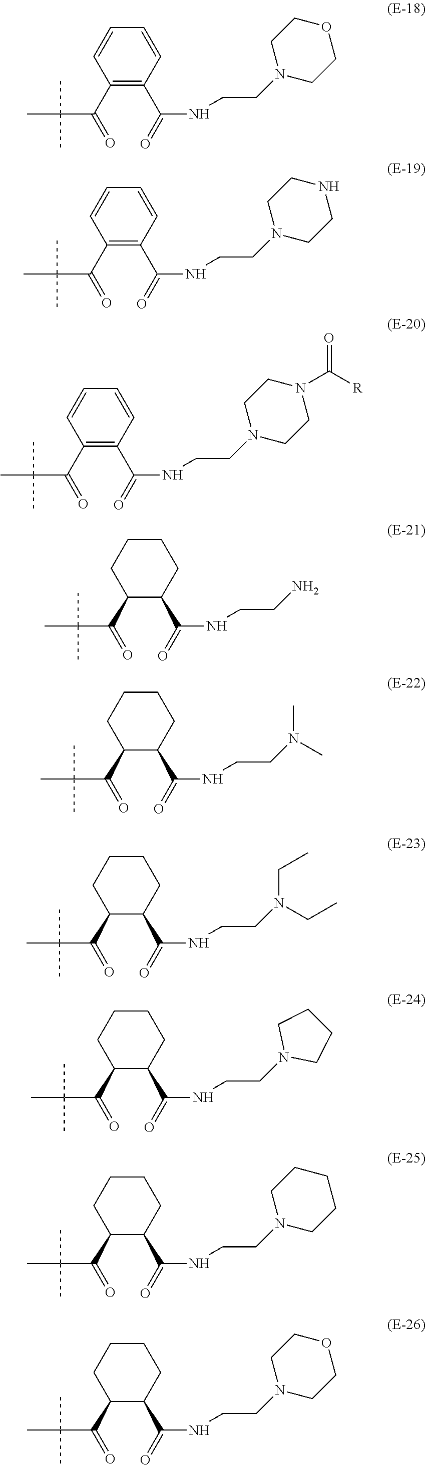

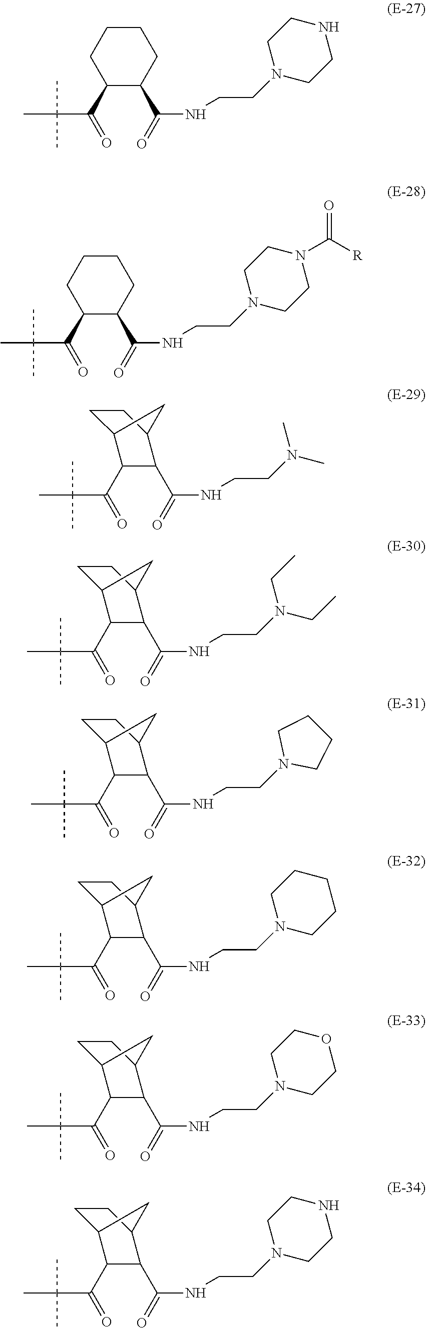

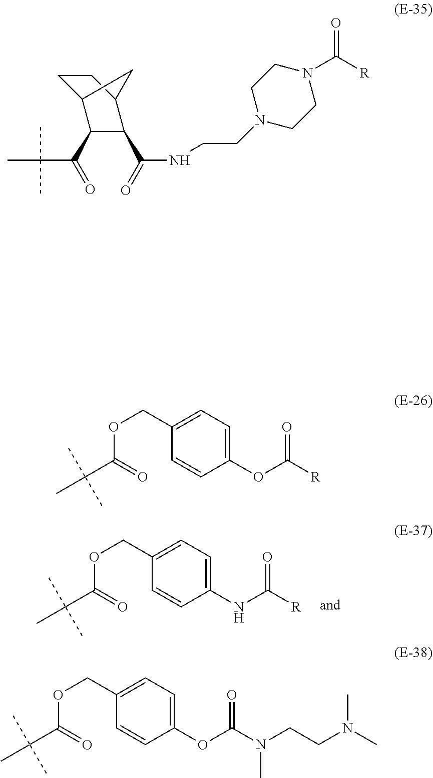

wherein the dashed line indicates the attachment to —W— of formula (I);
any one of the phenyl rings of the formulae (E-1) to (E-38) is optionally substituted with 1, 2, 3, 4 or 5 substituents selected from the group consisting of C₁₋₁₀ alkyl, —OH, —NO₂, —CN, halogen and acyl and wherein —R is C₁₋₁₀ alkyl.

In certain embodiments, any of the phenyl rings in the formula (E-1) to (E-38) is optionally substituted with 1, 2, 3 or 4 substituents selected from the group consisting of —F, —Cl, —CN, acetyl, —NO₂ and —CF₃.

In certain embodiments, -E is of formula (E-1) to (E-12).

In certain embodiments, -E is any one of the oligoamides described in the US Publication No. 2015/0057221, US Publication No. 2014/0249093, U.S. Pat. Nos. 8,377,917, 8,906,847, 9,173,953 or U.S. Pat. No. 9,062,094, which are herewith incorporated by reference.

In certain embodiments, -E is cleavable at physiological pH such as by β-elimination and is any of the β-eliminative moiety described for example in U.S. Pat. No. 9,387,245 or U.S. Pat. No. 8,754,190, which are herewith incorporated by reference.

In certain embodiments, —R³ and —R² together with the nitrogen to which —R³ is attached and the carbon atom to which —R² is attached form a ring selected from the group consisting of:

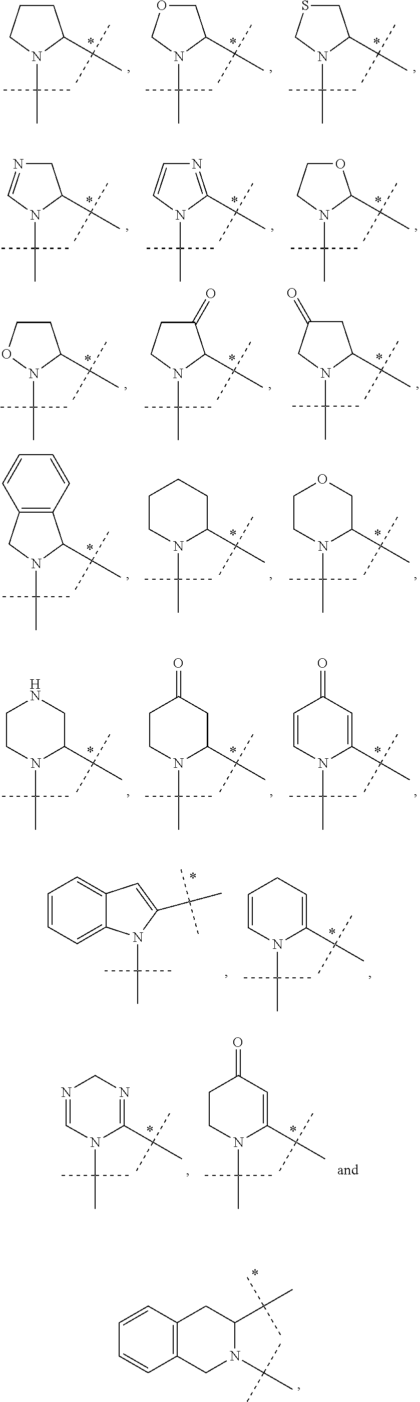

wherein the unmarked dashed line indicates the attachment to -E and the dashed line marked with an asterisk indicates the attachment to the carbon atom to which —R¹ is attached.

In certain embodiments, the moiety:

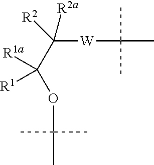

is an 2-amino alcohol, which is not serine or a derivative thereof, threonine or a derivative thereof, or cis-amino indanol or a derivative thereof. In certain embodiments, the 2-amino alcohol is not aminoethanol.

In certain embodiments, -L¹- is further substituted with one or more substituents.

In certain embodiments, -L¹- is not further substituted.

In certain embodiments, all moieties -L²- of the conjugate of the present invention are identical. In certain embodiments, the conjugate of the present invention comprises more than one type of -L²-, such as two, three, four or five different moieties -L²-. Such more than one type of -L²- may be connected to only one type of -L¹- or may be connected to more than one type of -L¹-.

In certain embodiments, -L²- is a chemical bond.

In certain embodiments, -L²- is a spacer moiety.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
—R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R⁵)(R^{y5a}), —S(O)N(R^{y5})(R^{y5a}), —S(O)₂R^{y5}, —S(O)R^{y5}, —N(R^{y5})S(O)₂N(R⁵)(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R⁵)(R^{y5a}), —OC(O)N(R^{y5})(R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R³)—, —S(O)N(R³)—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T′ is independently optionally substituted with one or more —R^{y2}, which are the same or different;
—R^{y2} is selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R⁵)(R^{y5a}), —S(O)N(R^{y5})(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y5b}), —OC(O)N(R^{y5})(R^{y5a}) and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{yla})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
—R^{y1} and —R^{y1a} are independently selected from the group consisting of —H, -T′, C₁₋₁₀ alkyl, C₂₋₁₀ alkenyl and C₂₋₁₀ alkynyl;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl;
each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from the group consisting of —O—, -T′- and —C(O)N(R^{y1})—; and which C₁₋₂₀ alkyl chain is optionally substituted with one or more groups independently selected from the group consisting of —OH, -T′ and —C(O)N(R^{y6}R^{y6a}); wherein —R^{y1}, —R^{y6}, —R^{y6}a are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²- has a molecular weight in the range of from 14 g/mol to 750 g/mol.

In certain embodiments, -L²- comprises a moiety selected from the group consisting of:

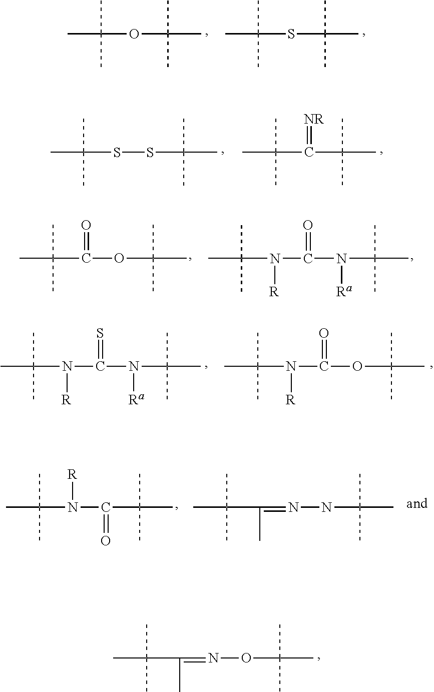

wherein dashed lines indicate attachment to -L¹-, the remainder of -L²- or Z, respectively; and —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, Z is a polymeric moiety.

In certain embodiments, Z is a C₈₋₂₄ alkyl.

In certain embodiments, Z is water-soluble.

In certain embodiments, Z is a water-soluble polymeric moiety.

If Z is a water-soluble polymeric moiety, such polymeric moiety has a molecular weight ranging from and including 1 kDa to 1000 kDa. In certain embodiments, Z has a molecular weight ranging from and including 5 kDa to 1000 kDa. In certain embodiments, Z has a molecular weight ranging from and including 5 kDa to 500 kDa. In certain embodiments, Z has a molecular weight ranging from and including 10 kDa to 250 kDa. In certain embodiments, Z has a molecular weight ranging from and including 10 kDa to 150 kDa. In certain embodiments, Z has a molecular weight ranging from and including 12 kDa to 100 kDa. In certain embodiments, Z has a molecular weight ranging from and including 15 kDa to 80 kDa. In certain embodiments, Z has a molecular weight ranging from and including 10 kDa to 80 kDa.

In certain embodiments, Z has a molecular weight of about 80 kDa. In certain embodiments, Z has a molecular weight of about 70 kDa. In certain embodiments, Z has a molecular weight of about 60 kDa. In certain embodiments, Z has a molecular weight of about 50 kDa. In certain embodiments, Z has a molecular weight of about 40 kDa. In certain embodiments, Z has a molecular weight of about 30 kDa. In certain embodiments, Z has a molecular weight of about 20 kDa. In certain embodiments, Z has a molecular weight of about 10 kDa. In certain embodiments, Z has a molecular weight of about 5 kDa.

In certain embodiments, Z is a water-soluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a water-soluble polymeric moiety comprising a protein, such as a protein selected from the group consisting of carboxyl-terminal peptide of the chorionic gonadotropin as described in US2012/0035101A1 which are herewith incorporated by reference; albumin; XTEN sequences as described in WO2011/123813A2 which are herewith incorporated by reference; proline/alanine random coil sequences as described in WO2011/144756A1 which are herewith incorporated by reference; proline/alanine/serine random coil sequences as described in WO2008/155134A1 and WO2013/024049A1 which are herewith incorporated by reference; and Fc-fusion proteins.

In certain embodiments, Z is a polysarcosine. In certain embodiments, Z comprises poly(N-methylglycine). In certain embodiments, Z comprises a random coil protein moiety.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine and proline. In certain embodiments, at least 10%, but less than 75%, in certain embodiments less than 65% of the total number of amino acid residues of such random coil protein moiety are proline residues. In certain embodiments, such random coil protein moiety is as described in WO2011/144756A1, which is hereby incorporated by reference in its entirety. In certain embodiments, Z comprises at least one moiety selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:51 and SEQ ID NO:61 as disclosed in WO2011/144756A1, which are hereby incorporated by reference. A moiety comprising such random coil protein comprising alanine and proline will be referred to as “PA” or “PA moiety”. Accordingly, in certain embodiments, Z comprises a PA moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine, serine and proline. In certain embodiments, at least 4%, but less than 40% of the total number of amino acid residues of such random coil protein moiety are proline residues. In certain embodiments, such random coil protein moiety is as described in WO2008/155134A1, which is hereby incorporated by reference in its entirety. In certain embodiments, Z comprises at least one moiety selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:40, SEQ ID NO:42, SEQ ID NO:44, SEQ ID NO:46, SEQ ID NO:50, SEQ ID NO:52, SEQ ID NO:54 and SEQ ID NO:56 as disclosed in WO 2008/155134 A1, which are hereby incorporated by reference. A moiety comprising such random coil protein moiety comprising alanine, serine and proline will be referred to as “PAS” or “PAS moiety”. Accordingly, in certain embodiments, Z comprises a PAS moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments 99% of the total number of amino acids forming said random coil protein moiety are selected from alanine, glycine, serine, threonine, glutamate and proline. In certain embodiments, such random coil protein moiety is as described in WO2010/091122A1 which is hereby incorporated by reference. In certain embodiments, Z comprises at least one moiety selected from the group consisting of SEQ ID NO:182, SEQ ID NO:183, SEQ ID NO:184; SEQ ID NO:185, SEQ ID NO:186, SEQ ID NO:187, SEQ ID NO:188, SEQ ID NO:189, SEQ ID NO:190, SEQ ID NO: 191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:1715, SEQ ID NO:1716, SEQ ID NO:1718, SEQ ID NO:1719, SEQ ID NO:1720, SEQ ID NO:1721 and SEQ ID NO:1722 as disclosed in WO2010/091122A1, which are hereby incorporated by reference. A moiety comprising such random coil protein moiety comprising alanine, glycine, serine, threonine, glutamate and proline will be referred to as “XTEN” or “XTEN moiety” in line with its designation in WO2010/091122A1. Accordingly, in certain embodiments, Z comprises an XTEN moiety.

In certain embodiments, Z is a hyaluronic acid-based polymer.

In certain embodiments, Z is a polymeric moiety as disclosed in WO2013/024047A1, which is herewith incorporated by reference. In certain embodiments, Z is a polymeric moiety as disclosed in WO2013/024048A1, which is herewith incorporated by reference.

In certain embodiments, Z is a PEG-based polymer, such as linear, branched or multi-arm PEG-based polymer.

In certain embodiments, Z is a linear PEG-based polymer.

In certain embodiments, Z is a branched C₈₋₂₄ alkyl having one, two, three, four, five or six branching points. In certain embodiments, Z is a branched C₈₋₂₄ alkyl having one, two or three branching points. In certain embodiments, Z is a branched C₈₋₂₄ alkyl having one branching point. In certain embodiments, Z is a branched C₈₋₂₄ alkyl having two branching points. In certain embodiments, Z is a branched C₈₋₂₄ alkyl having three branching points.

In certain embodiments, Z is a branched polymer. In certain embodiments, Z is a branched polymer having one, two, three, four, five or six branching points. In certain embodiments, Z is a branched polymer having one, two or three branching points. In certain embodiments, Z is a branched polymer having one branching point. In certain embodiments, Z is a branched polymer having two branching points. In certain embodiments, Z is a branched polymer having three branching points.

In certain embodiments, a branching point is selected from the group consisting of —N<, —CH< and >C<.

In certain embodiments, such branched moiety Z is PEG-based.

In certain embodiments, Z is a multi-arm PEG-based polymer.

In certain embodiments, Z is a multi-arm PEG-based polymer having at least 2 PEG-based arms, such as 2, 3, 4, 5, 6, 7, or 8 PEG-based arms.

In certain embodiments, Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, Z is water insoluble.

In certain embodiments, Z is a water-insoluble polymeric moiety.

In certain embodiments, Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a hydrogel.

In certain embodiments, Z is a PEG-based or hyaluronic acid-based hydrogel. In certain embodiments, Z is a PEG-based hydrogel. In certain embodiments, Z is a hyaluronic acid-based hydrogel.

In certain embodiments, Z is a hydrogel as described in WO2006/003014A2, WO2011/012715A1 or WO2014/056926A1, which are herewith incorporated by reference in their entirety.

In certain embodiments, Z is a hydrogel as disclosed in WO2013/036847A1. In particular, in certain embodiments, Z is a hydrogel produced by a method comprising the step of reacting at least a first reactive polymer with a cleavable crosslinker compound, wherein said cleavable crosslinker compound comprises a first functional group —Y¹ that reacts with the first reactive polymer and further comprises a moiety that is cleaved by elimination under physiological conditions wherein said moiety comprises a second functional group —Y² that reacts with a second reactive polymer. In certain embodiments, the cleavable crosslinker compound is of formula (PL-1):

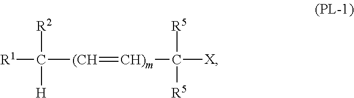

wherein
m is 0 or 1;
—X comprises a functional group capable of connecting to a reactive polymer that is amenable to elimination under physiological conditions and said second functional group —Y²;
at least one of —R¹, —R² and —R⁵ comprises said first functional group —Y¹ capable of connecting to a polymer;
one and only one of —R¹ and —R² is selected from the group consisting of —H, alkyl, arylalkyl, and heteroarylalkyl;
optionally, —R¹ and —R² may be joined to form a 3- to 8-membered ring;
at least one or both of —R¹ and —R² is independently selected from the group consisting of —CN, —NO₂, aryl, heteroaryl, alkenyl, alkynyl, —COR³, —SOR³, —SO₂R³ and —SR⁴;
—R³ is selected from the group consisting of —H, alkyl, aryl, arylalkyl, heteroaryl, heteroarylalkyl, —OR⁹ and —NR⁹₂;
—R⁴ is selected from the group consisting of alkyl, aryl, arylalkyl, heteroaryl and heteroarylalkyl;
each —R⁵ is independently selected from the group consisting of —H, alkyl, alkenylalkyl, alkynylalkyl, (OCH₂CH₂)ₚO-alkyl with p being an integer ranging from 1 to 1000, aryl, arylalkyl, heteroaryl and heteroarylalkyl;
each —R⁹ is independently selected from the group consisting of —H and alkyl or both —R⁹ together with the nitrogen to which they are attached form a heterocyclic ring; and wherein the moiety of formula (PL-1) is optionally further substituted.

The following paragraphs describe such hydrogel in more detail.

In certain embodiments, —X of formula (PL-1) is selected from the group consisting of succinimidyl carbonate, sulfosuccinimidyl carbonate halides, thioethers, esters, nitrophenyl carbonate, chloroformate, fluoroformate, optionally substituted phenols and formula (PL-2):

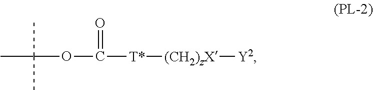

wherein
the dashed line indicates attachment to the remainder of formula (PL-1);
-T*- is selected from the group consisting of —O—, —S— and —NR⁶—;
z is an integer selected from the group consisting of 1, 2, 3, 4, 5 and 6;
—X′— is absent or is selected from the group consisting of —OR⁷— and —SR⁷—;
—Y² is a functional group capable of connecting with a reactive polymer;
—R⁶ is selected from the group consisting of —H, alkyl, aryl, heteroaryl, arylalkyl, and heteroarylalkyl; and
—R⁷ is selected from the group consisting of alkylene, phenylene and (OCH₂CH₂)ₚ, with p being an integer ranging from 1 to 1000.

In certain embodiments, —X of formula (PL-1) comprises an activated carbonate such as succinimidyl carbonate, sulfosuccinimidyl carbonate, or nitrophenyl carbonate. In certain embodiments, —X of formula (PL-1) comprises a carbonyl halide such as —O(C═O)Cl or O(C═O)F. In certain embodiments, —X of formula (PL-1) has the formula (PL-2). In certain embodiments, —X of formula (PL-1) is —OR⁷ or —SR⁷, wherein —R⁷ is optionally substituted alkylene, optionally substituted phenylene or (OCH₂CH₂)ₚ, wherein p is 1 to 1000.

In certain embodiments, p of formula (PL-2) is an integer ranging from 1 to 100. In certain embodiments, p of formula (PL-2) is an integer ranging from 1 to 10.

In certain embodiments, —Y¹ of formula (PL-1) and —Y² of formula (PL-2) independently comprise —N₃, —NH₂, —NH—CO₂^{t}Bu, —SH, —S^{t}Bu, maleimide, —CO₂H, —CO₂^{t}Bu, 1,3-diene, cyclopentadiene, furan, alkyne, cyclooctyne, acrylate or acrylamide, wherein ^{t}Bu is tert-butyl, and wherein when one of —Y¹ or —Y² comprises —N₃ the other does not comprise alkyne or cyclooctyne; when one of —Y¹ or —Y² comprises —SH the other does not comprise maleimide, acrylate or acrylamide; when one of —Y¹ or —Y² comprises —NH₂ the other does not comprise —CO₂H; when one of —Y¹ or —Y² comprises 1,3-diene or cyclopentadiene the other does not comprise furan.

In certain embodiments, the cleavable crosslinker compound is of formula (PL-3)

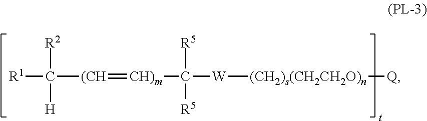

wherein
m is 0 or 1;
n is an integer selected from 1 to 1000;
s is 0, 1 or 2;
t is selected from the group consisting of 2, 4, 8, 16 and 32;
—W— is selected from the group consisting of —O(C═O)O—, —O(C═O)NH—, —O(C═O)S—, —O(C═O)NR⁶CH₂O— and —O(C═O)NR⁶S—;
-Q is a core group having a valency=t; which connects the multiple arms of the cleavable crosslinking compound,
wherein t is an integer selected from 2, 4, 8, 16 and 32, and
wherein —R¹, —R² and —R⁵ are defined as in formula (PL-1).

In certain embodiments, t of formula (PL-3) is 2. In certain embodiments, t of formula (PL-3) is 4. In certain embodiments, t of formula (PL-3) is 8. In certain embodiments, t of formula (PL-3) is 16. In certain embodiments, t of formula (PL-3) is 32.

In certain embodiments, -Q of formula (PL-3) has a structure selected from the group consisting of

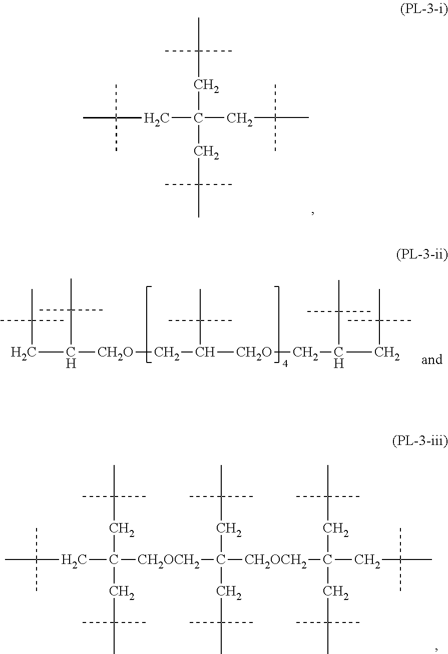

wherein the dashed lines indicate attachment to the remainder of the cleavable crosslinker compound.

In certain embodiments, -Q of formula (PL-3) has the structure of (PL-3-i). In certain embodiments, -Q of formula (PL-3) has the structure of (PL-3-ii). In certain embodiments, -Q of formula (PL-3) has the structure of (PL-3-iii).

In certain embodiments, the cleavable crosslinker compound is of formula (PL-3), wherein m is O, n is approximately 100, s is 0, t is 4, —W— is —O(C═O)NH—, -Q has the structure of (PL-3i), —R² is —H, one —R⁵ is —H and the other —R⁵ is —(CH₂)₅N₃, and —R¹ is (4-chlorophenyl)SO₂, phenyl substituted with —SO₂, morpholino-SO₂, or —CN.

In certain embodiments, —Y¹ of formula (PL-3) comprises —N₃, —NH₂, —NH—CO₂^{t}Bu, —SH, —S^{t}Bu, maleimide, —CO₂H, —CO₂^{t}Bu, 1,3-diene, cyclopentadiene, furan, alkyne, cyclooctyne, acrylate or acrylamide, wherein ^{t}Bu is tert-butyl.

In certain embodiments, each —Y¹ of formula (PL-1) or (PL-3) and —Y² of formula (PL-2) independently comprises —N₃, —NH₂, —NH—CO₂^{t}Bu, —SH, —S^{t}Bu, maleimide, —CO₂H, —CO₂^{t}Bu, 1,3-diene, cyclopentadiene, furan, alkyne, cyclooctyne, acrylate or acrylamide.

In certain embodiments, one of —Y¹ and —Y² is azide and the other is a reactive functional group selected from the group consisting of acetylene, cyclooctyne, and maleimide. In certain embodiments, one of —Y¹ and —Y² is thiol and the other is a reactive functional group selected from the group consisting of maleimide, acrylate, acrylamide, vinylsulfone, vinylsulfonamide, and halocarbonyl. In certain embodiments, one of —Y¹ and —Y² is amine and the other is a selective reactive functional group selected from carboxylic acid and activated carboxylic acid. In certain embodiments, one of —Y¹ and —Y² is maleimide and the other is a selective reactive functional group selected from the group consisting of 1,3-diene, cyclopentadiene, and furan.

In certain embodiments, the first and any second polymer is selected from the group consisting of homopolymeric or copolymeric polyethylene glycols, polypropylene glycols, poly(N-vinylpyrrolidone), polymethacrylates, polyphosphazenes, polylactides, polyacrylamides, polyglycolates, polyethylene imines, agaroses, dextrans, gelatins, collagens, polylysines, chitosans, alginates, hyaluronans, pectins and carrageenans that either comprise suitable reactive functionalities or is of formula [Y³—(CH₂)ₛ(CH₂CH₂O)ₙ]ₜQ, wherein —Y³ is a reactive functional group, s is 0, 1 or 2, n is an integer selected from the group ranging from 10 to 1000, -Q is a core group having valency t, and t is an integer selected from the group consisting of 2, 4, 8, 16 and 32.

In certain embodiments, the first polymer comprises a multi-arm polymer. In certain embodiments, the first polymer comprises at least three arms. In certain embodiments, the first polymer comprises at least four arms. In certain embodiments, the first polymer comprises at least five arms. In certain embodiments, the first polymer comprises at least six arms. In certain embodiments, the first polymer comprises at least seven arms. In certain embodiments, the first polymer comprises at least eight arms.

In certain embodiments, the second polymer comprises a multi-arm polymer. In certain embodiments, the second polymer comprises at least three arms. In certain embodiments, the second polymer comprises at least four arms. In certain embodiments, the second polymer comprises at least five arms. In certain embodiments, the second polymer comprises at least six arms. In certain embodiments, the second polymer comprises at least seven arms. In certain embodiments, the second polymer comprises at least eight arms.

In certain embodiments, the first polymer comprises a 2-arm polyethylene glycol polymer. In certain embodiments, the first polymer comprises a 4-arm polyethylene glycol polymer. In certain embodiments, the first polymer comprises an 8-arm polyethylene glycol polymer. In certain embodiments, the first polymer comprises a 16-arm polyethylene glycol polymer. In certain embodiments, the first polymer comprises a 32-arm polyethylene glycol polymer.

In certain embodiments, the second polymer comprises a 2-arm polyethylene glycol polymer. In certain embodiments, the second polymer comprises a 4-arm polyethylene glycol polymer. In certain embodiments, the second polymer comprises an 8-arm polyethylene glycol polymer. In certain embodiments, the second polymer comprises a 16-arm polyethylene glycol polymer. In certain embodiments, the second polymer comprises a 32-arm polyethylene glycol polymer.

In certain embodiments, the first and a second reactive polymer are reacted with said cleavable crosslinker compound, either sequentially or simultaneously.

In certain embodiments, the first and second functional groups are the same.

Only in the context of formulas (PL-1), (PL-2) and (PL-3) the terms used have the following meaning:

The term “a moiety capable of being cleaved by elimination under physiological conditions” refers to a structure comprising a group H—C—(CH═CH)ₘ—C—X′ wherein m is 0 or 1 and X′ is a leaving group, wherein an elimination reaction as described above to remove the elements of HX′ can occur at a rate such that the half-life of the reaction is between 1 and 10,000 hours under physiological conditions of pH and temperature. Preferably, the half-life of the reaction is between 1 and 5,000 hours, and more preferably between 1 and 1,000 hours, under physiological conditions of pH and temperature. By physiological conditions of pH and temperature is meant a pH of between 7 and 8 and a temperature between 30 and 40 degrees centigrade.

The term “reactive polymer and reactive oligomer” refers to a polymer or oligomer comprising functional groups that are reactive towards other functional groups, most preferably under mild conditions compatible with the stability requirements of peptides, proteins, and other biomolecules. Suitable functional groups found in reactive polymers include maleimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “functional group capable of connecting to a reactive polymer” refers to a functional group that reacts to a corresponding functional group of a reactive polymer to form a covalent bond to the polymer. Suitable functional groups capable of connecting to a reactive polymer include maleimides, thiols or protected thiols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “substituted” refers to an alkyl, alkenyl, alkynyl, aryl, or heteroaryl group comprising one or more substituent groups in place of one or more hydrogen atoms. Substituent groups may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched, and cyclic; amino, alkylamino, dialkylamino, silyl including alkylsilyl, alkoxysilyl, and arylsilyl; nitro; cyano; carbonyl; carboxylic acid, carboxylic ester, carboxylic amide; aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

The properties of —R¹ and —R² may be modulated by the optional addition of electron-donating or electron-withdrawing substituents. By the term “electron-donating group” is meant a substituent resulting in a decrease in the acidity of the R¹R²CH; electron-donating groups are typically associated with negative Hammett σ or Taft σ* constants and are well-known in the art of physical organic chemistry: Hammett constants refer to aryl/heteroaryl substituents, Taft constants refer to substituents on non-aromatic moieties. Examples of suitable electron-donating substituents include lower alkyl, lower alkoxy, lower alkylthio, amino, alkylamino, dialkylamino, and silyl.

The term “electron-withdrawing group” refers to a substituent resulting in an increase in the acidity of the R¹R²CH group; electron-withdrawing groups are typically associated with positive Hammett σ or Taft σ* constants and are well-known in the art of physical organic chemistry. Examples of suitable electron-withdrawing substituents include halogen, difluoromethyl, trifluoromethyl, nitro, cyano, C(═O)—R^{x}, wherein —R^{x} is H, lower alkyl, lower alkoxy, or amino, or S(O)ₘR^{Y}, wherein m is 1 or 2 and —R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett σ constant, while an alkoxy substituent on the meta-position of an aryl ring is electron-withdrawing and is characterized by a positive Hammett σ constant.

The terms “alkyl”, “alkenyl”, and “alkynyl” include linear, branched or cyclic hydrocarbon groups of 1 to 8 carbons or 1 to 6 carbons or 1 to 4 carbons wherein alkyl is a saturated hydrocarbon, alkenyl includes one or more carbon-carbon double bonds and alkynyl includes one or more carbon-carbon triple bonds. Unless otherwise specified these contain 1 to 6 carbons.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “halogen” includes fluoro, chloro, bromo and iodo.

The term “maleimido” is a group of the formula:

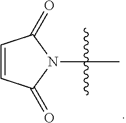 [SMILES: *N1C(=O)C=CC1=O]

In certain embodiments, Z is a hydrogel as disclosed in WO2020/206358A1. In particular, in certain embodiments, Z is a hydrogel produced by a method comprising the steps of:
(a) providing a first prepolymer comprising a multi-arm polymer —P², wherein said first prepolymer is of formula (PL-4)

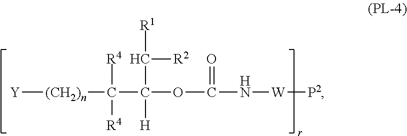

wherein
n is an integer selected from 0, 1, 2, 3, 4, 5 and 6;
r is an integer higher than 2;
—Y is a reactive functional group for connecting said first prepolymer to a second prepolymer;
—R¹ and —R² are independently an electron-withdrawing group, alkyl, or —H, and wherein at least one of —R¹ and —R² is an electron-withdrawing group;
each —R⁴ is independently C₁-C₃ alkyl or the two —R⁴ form together with the carbon atom to which they are attached a 3- to 6-membered ring;
—W— is absent or is

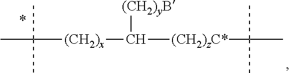

wherein the dashed line marked with the asterisk indicates the attachment to —NH— and the unmarked dashed line indicates the attachment to —P²;
each of x, y, and z is independently an integer selected from 0, 1, 2, 3, 4, 5 and 6;
—B′ is —NH₂, —ONH₂, ketone, aldehyde, —SH, —OH, —CO₂H, carboxamide group, or a group comprising a cyclooctyne or bicyclononyne; and
—C* is carboxamide, thioether, thiosuccinimidyl, triazole, or oxime;
(b) providing the second prepolymer comprising a multi-arm polymer —P¹ wherein each arm is terminated by a reactive functional group —Y″ that reacts with —Y of step (a);
(c) mixing the two prepolymers of steps (a) and (b) under conditions wherein —Y and —Y″ react to form a linkage —Y*—; and optionally
(d) isolating the resulting hydrogel.

Accordingly, —Z is a hydrogel obtainable from the method described above. In certain embodiments, the hydrogel produced by the preceding method is degradable.

In certain embodiments, —Y and —Y″ react under step (c) to form an insoluble hydrogel matrix comprising crosslinks of formula (PL-4′):

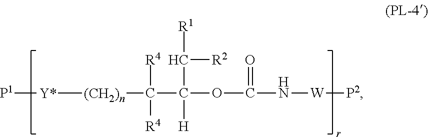

wherein n, r, —P¹, —Y*—, —R⁴, —R¹, —R², —W— and —P² are as defined above.

In certain embodiments, n of formula (PL-4) or (PL-4′) is an integer selected from 1, 2, 3, 4, 5 and 6. In certain embodiments, n of formula (PL-4) or (PL-4′) is an integer selected from 1, 2 and 3. In certain embodiments, n of formula (PL-4) or (PL-4′) is an integer selected from 0, 1, 2 and 3. In certain embodiments, n of formula (PL-4) or (PL-4′) is 1. In certain embodiments, n of formula (PL-4) is 2. In certain embodiments, n of formula (PL-4) or (PL-4′) is 3.

In certain embodiments, the multi-arm —P² of formula (PL-4) or (PL-4′) is an r-armed polymer, wherein r is an integer selected from 2, 3, 4, 5, 6, 7, 8, 9, 10, 11 and 12. In certain embodiments, r of formula (PL-4) or (PL-4′) is an integer selected from 2, 3, 4, 5, 6, 7 and 8. In certain embodiments, r of formula (PL-4) or (PL-4′) is an integer selected from 2, 4, 6 and 8. In certain embodiments, r of formula (PL-4) or (PL-4′) is 2. In certain embodiments r of formula (PL-4) or (PL-4′) is 4. In certain embodiments, r of formula (PL-4) or (PL-4′) is 6. In certain embodiments, r of formula (PL-4) or (PL-4′) is 8.

In certain embodiments, —P² of formula (PL-4) or (PL-4′) has a molecular weight of at least 1 kDa. In certain embodiments, —P² of formula (PL-4) or (PL-4′) has a molecular weight of 1 to 100 kDa. In certain embodiments, —P² of formula (PL-4) or (PL-4′) has a molecular weight of 1 to 80 kDa. In certain embodiments, —P² of formula (PL-4) or (PL-4′) has a molecular weight of 1 to 60 kDa. In certain embodiments, —P² of formula (PL-4) or (PL-4′) has a molecular weight of 1 to 40 kDa. In certain embodiments, —P² of formula (PL-4) or (PL-4′) has a molecular weight of 1 to 20 kDa. In certain embodiments, —P² of formula (PL-4) or (PL-4′) has a molecular weight of 1 to 10 kDa. In certain embodiments, —P² of formula (PL-4) or (PL-4′) has a molecular weight of 1 to 5 kDa. In certain embodiments, —P² of formula (PL-4) or (PL-4′) has a molecular weight of about 20 kDa. In certain embodiments, —P² of formula (PL-4) or (PL-4′) has a molecular weight of about 40 kDa. In certain embodiments, —P² of formula (PL-4) or (PL-4′) has a molecular weight of about 60 kDa. In certain embodiments, —P² of formula (PL-4) or (PL-4′) has a molecular weight of about 80 kDa.

In certain embodiments, the multi-arm polymer —P¹ of step (b) is an r-armed polymer, wherein r is an integer selected from 2, 3, 4, 5, 6, 7, 8, 9, 10, 11 and 12. In certain embodiments, the multi-arm —P¹ of step (b) is an r-armed polymer, wherein r is an integer selected from 2, 3, 4, 5, 6, 7 and 8. In certain embodiments, the multi-arm —P¹ of step (b) is an r-armed polymer, wherein r is an integer selected from 2, 4, 6 and 8. In certain embodiments, the multi-arm —P¹ of step (b) is an r-armed polymer, wherein r is 2. In certain embodiments, the multi-arm —P¹ of step (b) is an r-armed polymer, wherein r is 4. In certain embodiments, the multi-arm —P¹ of step (b) is an r-armed polymer, wherein r is 6. In certain embodiments, the multi-arm —P¹ of step (b) is an r-armed polymer, wherein r is 8.

In certain embodiments, —P¹ of step (b) has a molecular weight of at least 1 kDa. In certain embodiments, the multi-arm polymer —P¹ of step (b) has a molecular weight of 1 to 100 kDa. In certain embodiments, the multi-arm polymer —P¹ of step (b) has a molecular weight of 1 to 80 kDa. In certain embodiments, the multi-arm polymer —P¹ of step (b) has a molecular weight of 1 to 60 kDa. In certain embodiments, the multi-arm polymer —P¹ of step (b) has a molecular weight of 1 to 40 kDa. In certain embodiments, the multi-arm polymer —P¹ of step (b) has a molecular weight of 1 to 20 kDa. In certain embodiments, the multi-arm polymer —P¹ of step (b) has a molecular weight of 1 to 10 kDa. In certain embodiments, the multi-arm polymer —P¹ of step (b) has a molecular weight of 1 to 5 kDa. In certain embodiments, the multi-arm polymer —P¹ of step (b) has a molecular weight of about 20 kDa. In certain embodiments, the multi-arm polymer —P¹ of step (b) has a molecular weight of about 40 kDa. In certain embodiments, the multi-arm polymer —P¹ of step (b) has a molecular weight of about 60 kDa. In certain embodiments, the multi-arm polymer —P¹ of step (b) has a molecular weight of about 80 kDa.

In certain embodiments, —P¹ of step (b) and —P² of formula (PL-4) or (PL-4′) comprise poly(ethylene glycol) (PEG), poly(ethylene oxide) (PEO), poly(ethylene imine) (PEI), dextrans, hyaluronic acids, or co-polymers thereof. In certain embodiments, —P¹ of step (b) and P² of formula (PL-4) or (PL-4′) are PEG-based polymers. In certain embodiments, —P¹ of step (b) and —P² of formula (PL-4) or (PL-4′) are hyaluronic acid-based polymers.

In certain embodiments, —R¹ and —R² of formula (PL-4) or (PL-4′) are independently electron-withdrawing groups, alkyl, or —H, and wherein at least one of —R¹ and —R² is an electron-withdrawing group.

In certain embodiments, the electron-withdrawing group of —R¹ and —R² of formula (PL-4) or (PL-4′) is —CN, —NO₂, optionally substituted aryl, optionally substituted heteroaryl, optionally substituted alkenyl, optionally substituted alkynyl, —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments, the electron-withdrawing group of —R¹ and —R² of formula (PL-4) or (PL-4′) is —CN. In certain embodiments, the electron-withdrawing group of —R¹ and —R² of formula (PL-4) or (PL-4′) is —NO₂. In certain embodiments, the electron-withdrawing group of —R¹ and —R² of formula (PL-4) or (PL-4′) is optionally substituted aryl containing 6 to 10 carbons. In certain embodiments, the electron-withdrawing group of —R¹ and —R² of formula (PL-4) or (PL-4′) is optionally substituted phenyl, naphthyl, or anthracenyl. In certain embodiments the electron-withdrawing group of —R¹ and —R² of formula (PL-4) or (PL-4′) is optionally substituted heteroaryl comprising 3 to 7 carbons and containing at least one N, O, or S atom. In certain embodiments, the electron-withdrawing group of —R¹ and —R² of formula (PL-4) or (PL-4′) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments, the electron-withdrawing group of —R¹ and —R² of formula (PL-4) or (PL-4′) is optionally substituted alkenyl containing 2 to 20 carbon atoms. In certain embodiments, the electron-withdrawing group of —R¹ and —R² of formula (PL-4) or (PL-4′) is optionally substituted alkynyl containing 2 to 20 carbon atoms. In certain embodiments, the electron-withdrawing group of —R¹ and —R² of formula (PL-4) or (PL-4′) is —COR³, —SOR³, or —SO₂R³, wherein R³ is —H, optionally substituted alkyl containing 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl containing 1 to 20 carbon atoms, or both —R⁸ groups are taken together with the nitrogen to which they are attached to form a heterocyclic ring. In certain embodiments, the electron-withdrawing group of —R¹ and —R² of formula (PL-4) or (PL-4′) is —SR⁹, wherein —R⁹ is optionally substituted alkyl containing 1 to 20 carbon atoms, optionally substituted aryl, optionally substituted arylalkyl, optionally substituted heteroaryl, or optionally substituted heteroarylalkyl. In certain embodiments, at least one of —R¹ and —R² is —CN or —SO₂R³.

In certain embodiments, at least one of —R¹ and —R² of formula (PL-4) or (PL-4′) is —CN, —SOR³ or —SO₂R³. In certain embodiments, at least one of —R¹ and —R² of formula (PL-4) or (PL-4′) is —CN or —SO₂R³. In certain embodiments, at least one of —R¹ and —R² of formula (PL-4) or (PL-4′) is —CN or —SO₂R³, wherein —R³ is optionally substituted alkyl, optionally substituted aryl, or —NR⁸₂. In certain embodiments, at least one of —R¹ and —R² of formula (PL-4) or (PL-4′) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂. In certain embodiments, each —R⁴ of formula (PL-4) or (PL-4′) is independently C₁-C₃ alkyl or taken together may form a 3- to 6-membered ring. In certain embodiments, each —R⁴ of formula (PL-4) or (PL-4′) is independently C₁-C₃ alkyl. In certain embodiments, both —R⁴ of formula (PL-4) or (PL-4′) are methyl.

In certain embodiments, —Y and —Y″ are independently selected from the group consisting of amine, aminooxy, ketone, aldehyde, maleimidyl, thiol, alcohol, azide, 1,2,4,6-tetrazinyl, trans-cyclooctenyl, bicyclononynyl, cyclooctynyl, and protected variants thereof.

In certain embodiments, —Y and —Y″ may react with each other such as in a selective way. For example, when —Y is amine, —Y″ is carboxylic acid, active ester, or active carbonate to yield a residual connecting functional group —Y*— that is amide or carbamate. As another example, when —Y is azide, —Y″ is alkynyl, bicyclononynyl, or cyclooctynyl to yield a residual connecting functional group —Y*— that is 1,2,3-triazole. As another example, when —Y is NH₂O, —Y″ is ketone or aldehyde to yield a residual connecting functional group —Y*— that is oxime. As another example, when —Y is SH, —Y″ is maleimide or halocarbonyl to yield a residual connecting functional group —Y*— that is thiosuccinimidyl or thioether. Similarly, these roles of —Y and —Y″ can be reversed to yield —Y*— of opposing orientation.

In certain embodiments, —Y*— comprises an amide, oxime, 1,2,3-triazole, thioether, thiosuccinimide, or ether. In certain embodiments, —Y*— is -L²-.

These conjugation reactions may be performed under conditions known in the art, for example when —Y is azide and —Y″ is cyclooctyne the conjugation occurs in any solvent wherein both components show adequate solubility, although it is known that aqueous solutions show more favorable reaction rates. When mixed in an appropriate solvent, typically an aqueous buffer at a pH of 2 to 7 when —Y and —Y″ are azide/cyclooctyne, or at a pH of 6 to 9 when —Y and —Y″ are an activated ester and an amine, the —Y and —Y″ groups react to form an insoluble hydrogel matrix comprising crosslinks of formula (PL-4′). This process may be carried out in bulk phase, or under conditions of emulsification in a mixed organic/aqueous system so as to form microparticle suspensions such as microspheres that are suitable for injection. Only in the context of formulas (PL-4) and (PL-4′) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments, an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments, an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments, alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments, a heterocyclic ring or heterocyclyl is non-aromatic. In certain embodiments, a heterocyclic ring or heterocyclyl is aromatic.

The term “optionally substituted” refers to a group that may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR—R^{bb}, —NR^{aa}C(O)R^{bb}, —NR^{aa}C(O)OR^{bb}, —S(O)R^{aa}, —S(O)₂R^{aa}, —NR'S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR—R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments, Z is a polymer network formed through the physical aggregation of polymer chains, which physical aggregation is preferably caused by hydrogen bonds, crystallization, helix formation or complexation. In certain embodiments, such polymer network is a thermogelling polymer.

In certain embodiments, Z comprises a moiety selected from the group consisting of:

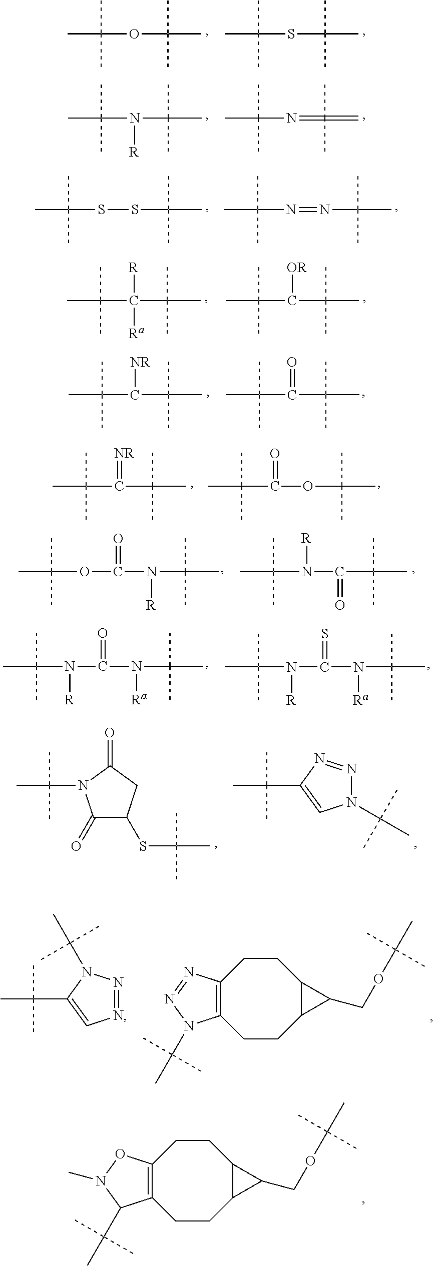

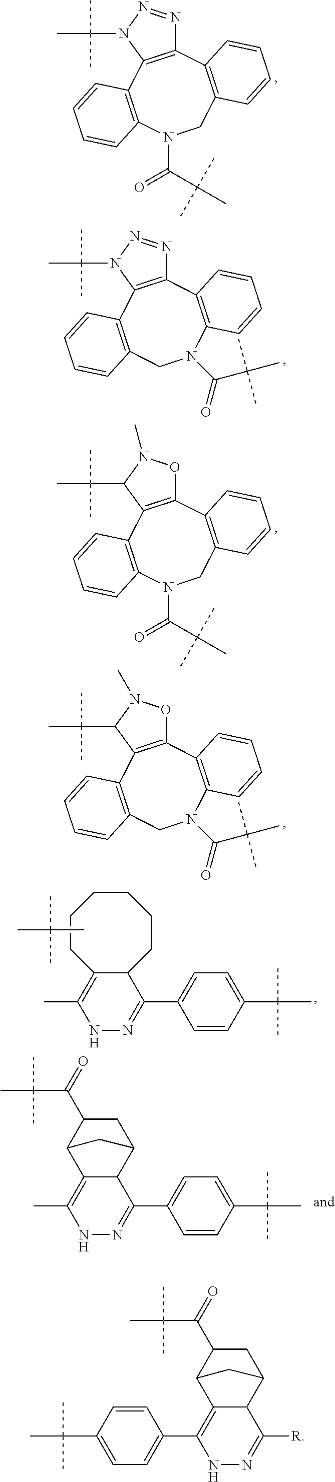

In certain embodiments, the conjugate of the present invention or the pharmaceutically acceptable salt thereof is of formula (Ia), (Ib), (Ic) or (Id):

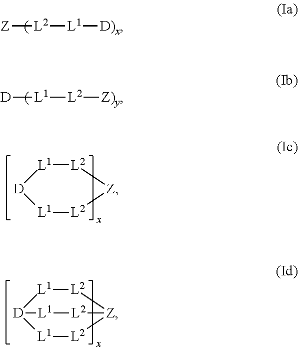

wherein
each -D, -L²- and Z are defined as above and each -L¹- is independently of formula (I);
x is an integer of at least 1; and
y is an integer selected from the group consisting of 2, 3, 4 and 5.

It is understood that even though one -D can be conjugated to multiple -L¹- moieties, the drug moiety is represented by “-D” and the drug by “D-H”.

In certain embodiments, the conjugate is of formula (Ia), (Ic) or (Id) and Z is a hydrogel. In such cases, a plurality of moieties -L²-L¹-D are conjugated to Z and it is understood that no upper limit for x can be provided.

In certain embodiments, the conjugate is of formula (Ia). In certain embodiments, the conjugate is of formula (Ib). In certain embodiments, the conjugate is of formula (Ic). In certain embodiments, the conjugate is of formula (Id). In certain embodiments, the conjugate is of formula (Ia) and Z is a hydrogel.

In certain embodiments, the conjugate is of formula (Ia), (Ic) or (Id), Z is a water-soluble polymeric moiety and x ranges from 2 to 1000, such as from 2 to 1500, such as from 2 to 1000, such as from 2 to 500, such as from 2 to 250 or such as from 2 to 100. In certain embodiments, the conjugate is of formula (Ia), (Ic) or (Id), Z is a water-soluble polymeric moiety and x is 20.

In certain embodiments, the conjugate is of formula (Ia), (Ic) or (Id), Z is a water-soluble polymeric moiety and x is 19. In certain embodiments, the conjugate is of formula (Ia), (Ic) or (Id), Z is a water-soluble polymeric moiety and x is 18. In certain embodiments, the conjugate is of formula (Ia), (Ic) or (Id), Z is a water-soluble polymeric moiety and x is 17. In certain embodiments, the conjugate is of formula (Ia), (Ic) or (Id), Z is a water-soluble polymeric moiety and x is 16. In certain embodiments, the conjugate is of formula (Ia), (Ic) or (Id), Z is a water-soluble polymeric moiety and x is 15. In certain embodiments, the conjugate is of formula (Ia), (Ic) or (Id), Z is a water-soluble polymeric moiety and x is 14. In certain embodiments, the conjugate is of formula (Ia), (Ic) or (Id), Z is a water-soluble polymeric moiety and x is 13. In certain embodiments, the conjugate is of formula (Ia), (Ic) or (Id), Z is a water-soluble polymeric moiety and x is 12. In certain embodiments, the conjugate is of formula (Ia), (Ic) or (Id), Z is a water-soluble polymeric moiety and x is 11. In certain embodiments, the conjugate is of formula (Ia), (Ic) or (Id), Z is a water-soluble polymeric moiety and x is 10. In certain embodiments, the conjugate is of formula (Ia), (Ic) or (Id), Z is a water-soluble polymeric moiety and x is 9. In certain embodiments, the conjugate is of formula (Ia), (Ic) or (Id), Z is a water-soluble polymeric moiety and x is 8. In certain embodiments, the conjugate is of formula (Ia), (Ic) or (Id), Z is a water-soluble polymeric moiety and x is 7. In certain embodiments, the conjugate is of formula (Ia), (Ic) or (Id), Z is a water-soluble polymeric moiety and x is 6. In certain embodiments, the conjugate is of formula (Ia), (Ic) or (Id), Z is a water-soluble polymeric moiety and x is 5. In certain embodiments, the conjugate is of formula (Ia), (Ic) or (Id), Z is a water-soluble polymeric moiety and x is 4. In certain embodiments, the conjugate is of formula (Ia), (Ic) or (Id), Z is a water-soluble polymeric moiety and x is 3. In certain embodiments, the conjugate is of formula (Ia), (Ic) or (Id), Z is a water-soluble polymeric moiety and x is 2.

In certain embodiments, the conjugate is of formula (Ib), Z is a water-soluble polymeric moiety and y is 1. In certain embodiments, the conjugate is of formula (Ib), Z is a water-soluble polymeric moiety and y is 2. In certain embodiments, the conjugate is of formula (Ib), Z is a water-soluble polymeric moiety and y is 3. In certain embodiments, the conjugate is of formula (Ib), Z is a water-soluble polymeric moiety and y is 4. In certain embodiments, the conjugate is of formula (Ib), Z is a water-soluble polymeric moiety and y is 5.

In certain embodiments, -L¹- of formula (I) is of formula (I′a):

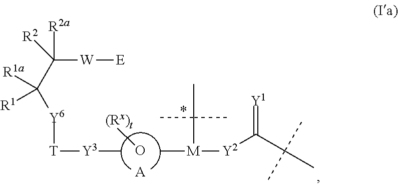

wherein the dashed line marked with the asterisk indicates the attachment to -L²- of formula (I) and the unmarked dashed line indicates the attachment to -D of formula (I);
t is selected from the group consisting of 1, 2, 3, 4, 5, 6, 7, 8, 9 and 10; and
—Y¹, —Y²—, -M-, -A-, —Y³—, -T-, —Y⁶—, —W—, -E, —R¹, —R^{1a}, —R², —R^{2a} and —R^{X} are used as defined in formula (I).

In certain embodiments, -L¹- of formula (I) is of formula (I′b):

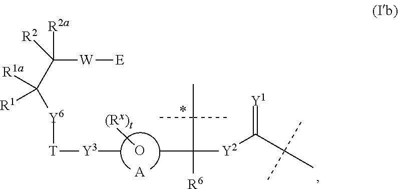

wherein the dashed line marked with the asterisk indicates the attachment to -L²- of formula (I) and the unmarked dashed line indicates the attachment to -D of formula (I);
t is selected from the group consisting of 1, 2, 3, 4, 5, 6, 7, 8, 9 and 10; and
═Y¹, —Y²—, -A-, —Y³—, -T-, —Y⁶—, —W—, -E, —R¹, —R^{1a}, —R², —R^{2a}, —R^{X} and —R⁶ are used as defined in formula (I).

In certain embodiments, -L¹- of formula (I) is of formula (I′c):

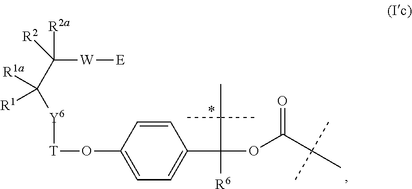

wherein the dashed line marked with the asterisk indicates the attachment to -L²- of formula (I) and the unmarked dashed line indicates the attachment to -D of formula (I) and -T-, —Y⁶—, —W—, -E, —R¹, —R^{1a}, —R², —R^{2a} and —R⁶ are used as defined in formula (I).

In certain embodiments, -L¹- of formula (I) is of formula (I′d):

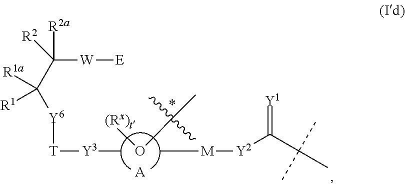

wherein the dashed line marked with the asterisk indicates the attachment to -L²- of formula (I) and the unmarked dashed line indicates the attachment to -D of formula (I);
t′ is selected from the group consisting of 1, 2, 3, 4, 5, 6, 7, 8 and 9; and
═Y¹, —Y²—, -M-, -A-, —Y³—, -T-, —Y⁶—, —W—, -E, —R¹, —R^{1a}, —R², —R^{2a} and —R^{X} are used as defined in formula (I).

In certain embodiments, -L¹- of formula (I) is of formula (I′e):

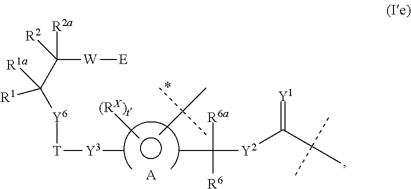

wherein the dashed line marked with the asterisk indicates the attachment to -L²- of formula (I) and the unmarked dashed line indicates the attachment to -D of formula (I);
t′ is selected from the group consisting of 1, 2, 3, 4, 5, 6, 7, 8 and 9; and
—Y¹, —Y²—, -A-, —Y³—, -T-, —Y⁶—, —W—, -E, —R¹, —R^{1a}, —R², —R^{2a}, —R^{X}, —R⁶ and —R^{6a} are used as defined in formula (I).

In certain embodiments, -L¹- of formula (I) is of formula (I′f):

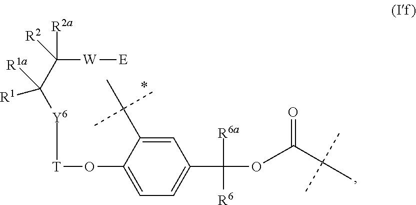

wherein the dashed line marked with the asterisk indicates the attachment to -L²- of formula (I), the unmarked dashed line indicates the attachment to -D of formula (I) and -T-, —Y⁶—, —W—, -E, —R¹, —R^{1a}, —R², —R^{2a}, —R⁶ and —R^{6a} are used as defined in formula (I).

In certain embodiments, -L¹- is of formula (I′a), (I′b), (I′c), (I′d), (I′e) or (I′f) and -D is selected from the group consisting of a primary or secondary amine-comprising drug moiety and a hydroxyl-comprising drug moiety. In certain embodiments, -L¹- is of formula (I′a), (I′b), (I′c), (I′d), (I′e) or (I′f) and -D is a primary or secondary amine-comprising drug moiety. In certain embodiments, -L¹- is of formula (I′a), (I′b), (I′c), (I′d), (I′e) or (I′f) and -D is a hydroxyl-comprising drug moiety. In certain embodiments, -L¹- is of formula (I′a), (I′b), (I′c), (I′d), (I′e) or (I′f) and -D is selected from the group consisting of a primary or secondary amine-comprising drug moiety and a hydroxyl-comprising drug moiety. In certain embodiments, -L¹- is of formula (I′a), (I′b), (I′c), (I′d), (I′e) or (I′f) and -D is a primary or secondary amine-comprising drug moiety. In certain embodiments, -L¹- is of formula (I′a), (I′b), (I′c), (I′d), (I′e) or (I′f) and -D is a hydroxyl-comprising drug moiety. In certain embodiments, -L¹- is of formula (I′a), (I′b), (I′c), (I′d), (I′e) or (I′f) and -D is selected from the group consisting of a primary or secondary amine-comprising drug moiety and a hydroxyl-comprising drug moiety. In certain embodiments, -L¹- is of formula (I′a), (I′b), (I′c), (I′d), (I′e) or (I′f) and -D is a primary or secondary amine-comprising drug moiety. In certain embodiments, -L¹- is of formula (I′a), (I′b), (I′c), (I′d), (I′e) or (I′f) and -D is a hydroxyl-comprising drug moiety.

Another aspect of the present invention is a conjugate or a pharmaceutically acceptable salt thereof comprising at least one moiety -D⁺ conjugated via at least one moiety -L¹-L²- to at least one moiety Z, wherein a moiety -L¹- is conjugated to a N⁺ of a moiety -D⁺ and wherein the linkage between -D⁺ and -L¹- is reversible and wherein a moiety -L²- is conjugated to Z, wherein
each -D⁺ is independently an electron-donating heteroaromatic N⁺-comprising moiety or a quaternary ammonium cation comprising moiety of a drug D, wherein each D comprises an electron-donating heteroaromatic N or a tertiary amine;
each -L²- is independently a single bond or a spacer moiety;
each Z is independently a polymeric moiety or a C₈₋₂₄ alkyl;
each -L¹- is independently a linker moiety of formula (IIa):

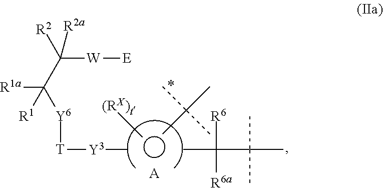

wherein the dashed line marked with the asterisk indicates the attachment to -L²- of formula (I) and the unmarked dashed line indicates the attachment to -D⁺;
t′ is selected from the group consisting of 1, 2, 3, 4, 5, 6, 7, 8 and 9; and
-A-, —Y³—, —Y⁶—, -T-, —W—, -E, —R¹, —R^{1a}, —R², —R^{2a}, —R^{x}, —R⁶ and —R^{6a} are used as defined in formula (I).

In certain embodiments, t′ of formula (I′d), (I′e) or (IIa) is selected from the group consisting of 1, 2, 3, 4, 5, 6 and 7. In certain embodiments, t′ of formula (I′d), (I′e) or (IIa) is selected from the group consisting of 1, 2, 3, 4, 5 and 6. In certain embodiments, t′ of formula (I′d), (I′e) or (IIa) is 1. In certain embodiments, t′ of formula (I′d), (I′e) or (IIa) is 2. In certain embodiments, t′ of formula (I′d), (I′e) or (IIa) is 3. In certain embodiments, t′ of formula (I′d), (I′e) or (IIa) is 4.

Another aspect of the present invention is a conjugate or a pharmaceutically acceptable salt thereof comprising at least one moiety -D⁺ conjugated via at least one moiety -L¹-L²- to at least one moiety Z, wherein a moiety -L¹- is conjugated to a N⁺ of a moiety -D⁺ and wherein the linkage between -D⁺ and -L¹- is reversible and wherein a moiety -L²- is conjugated to Z, wherein
each -D⁺ is independently an electron-donating heteroaromatic N⁺-comprising moiety or a quaternary ammonium cation comprising moiety of a drug D, wherein each D comprises an electron-donating heteroaromatic N or a tertiary amine;
each -L²- is independently a single bond or a spacer moiety;
each Z is independently a polymeric moiety or a C₈₋₂₄ alkyl;
each -L¹- is independently a linker moiety of formula (IIb):

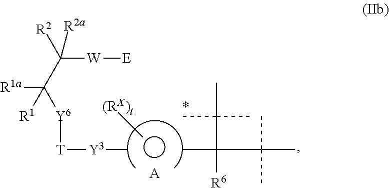

wherein the dashed line marked with the asterisk indicates the attachment to -L²- of formula (I) and the unmarked dashed line indicates the attachment to -D⁺;
t is selected from the group consisting of 1, 2, 3, 4, 5, 6, 7, 8, 9 and 10; and
-A-, —Y³—, -T-, —Y⁶—, —W—, -E, —R¹, —R^{1a}, —R², —R^{2a}, —R and —R⁶ are used as defined in formula (I).

In certain embodiments, t of formula (I′a), (I′b) or (IIb) is selected from the group consisting of 1, 2, 3, 4, 5, 6, 7 and 8. In certain embodiments, t of formula (I′a), (I′b) or (IIb) is selected from the group consisting of 1, 2, 3, 4, 5 and 6. In certain embodiments, t of formula (I′a), (I′b) or (IIb) is 1. In certain embodiments, t of formula (I′a), (I′b) or (IIb) is 2. In certain embodiments, t of formula (I′a), (I′b) or (IIb) is 3. In certain embodiments, t of formula (I′a), (I′b) or (IIb) is 4.

As used herein, the term “an electron-donating heteroaromatic N⁺-comprising moiety or a quaternary ammonium cation comprising moiety” refers to the moiety which after cleavage of the linkage between -D⁺ and -L¹- results in a drug D and wherein the drug moiety -D⁺ comprises at least one, such as one, two, three, four, five, six, seven, eight, nine or ten electron-donating heteroaromatic N⁺ or quaternary ammonium cations and analogously the corresponding D comprises at least one, such as one, two, three, four, five, six, seven, eight, nine or ten electron-donating heteroaromatic N or tertiary amines. Examples of chemical structures including heteroaromatic nitrogens, i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic l-system is marked with “§”:

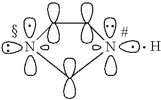

Such electron-donating heteroaromatic nitrogen atoms do not comprise heteroaromatic nitrogen atoms which donate one electron pair (i.e. not one electron) to the aromatic l-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

The conjugates of the present invention release one or more types of drug over an extended period of time, i.e. they are sustained-release conjugates. In certain embodiments, the release occurs with a release half-life ranging between 1 day and 2 months. In certain embodiments, the release occurs with a release half-life ranging between 1 day and 1 month. In certain embodiments, the release occurs with a release half-life ranging between 1 day and 20 days. In certain embodiments, the release occurs with a release half-life between 1 day and 15 days. In certain embodiments the release half-life may also range from 2 to 20 days, 4 to 15 days or 3 to 6 days.

Another aspect of the present invention is a pharmaceutical composition comprising at least one conjugate of the present invention or a pharmaceutical salt thereof.

In certain embodiments, the pharmaceutical composition comprises one conjugate of the present invention or a pharmaceutical salt thereof. In certain embodiments, the pharmaceutical composition comprises two conjugates of the present invention. In certain embodiments, the pharmaceutical composition comprises three conjugates of the present invention.

Such pharmaceutical composition may have a pH ranging from pH 3 to pH 8, such as ranging from pH 4 to pH 7 or ranging from pH 4 to pH 5. In certain embodiments, the pH of the pharmaceutical composition is about 4. In certain embodiments, the pH of the pharmaceutical composition is about 4.5. In certain embodiments, the pH of the pharmaceutical composition is about 5. In certain embodiments, the pH of the pharmaceutical composition is about 5.5. In certain embodiments, the pH of the pharmaceutical composition is about 6. In certain embodiments, the pH of the pharmaceutical composition is about 6.5. In certain embodiments, the pH of the pharmaceutical composition is about 7. In certain embodiments, the pH of the pharmaceutical composition is about 7.5.

In certain embodiments, the pH of the pharmaceutical composition is 4. In certain embodiments, the pH of the pharmaceutical composition is 4.5. In certain embodiments, the pH of the pharmaceutical composition is 5. In certain embodiments, the pH of the pharmaceutical composition is 5.5. In certain embodiments, the pH of the pharmaceutical composition is 6. In certain embodiments, the pH of the pharmaceutical composition is 6.5. In certain embodiments, the pH of the pharmaceutical composition is 7. In certain embodiments, the pH of the pharmaceutical composition is 7.5.

In certain embodiments, such pharmaceutical composition is a suspension formulation.

In certain embodiments, such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of
(i) Buffering agents: physiologically tolerated buffers to maintain pH in a desired range, such as sodium phosphate, bicarbonate, succinate, histidine, citrate, acetate, sulfate, nitrate, chloride, or pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs;
(ix) Anti-agglomeration agents, such as propylene glycol; and
(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect, the present invention relates to a conjugate of the present invention or a pharmaceutical composition comprising a conjugate of the present invention for use as a medicament.

In another aspect, the present invention relates to a conjugate or a pharmaceutically acceptable salt thereof of the present invention or a pharmaceutical composition comprising a conjugate of the present invention for use in a method of treating a disease that can be treated with D-H or its pharmaceutically acceptable salt thereof.

In a further aspect, the present invention relates to a method of preventing a disease or treating a patient suffering from a disease that can be prevented or treated with D-H comprising administering an effective amount of the conjugate or its pharmaceutically acceptable salt thereof of the present invention or the pharmaceutical compositions comprising said conjugates to the patient.

### Materials and Methods

Chemicals

All materials are obtained from commercial vendors except where stated otherwise.

Reactions

Reactions are performed with dry solvents (CH₂Cl₂, diethyl ether, DMF, THF, acetonitrile, pyridine) purchased from Sigma-Aldrich Chemie GmbH, Munich, Germany. Generally, reactions are stirred at room temperature and monitored by LCMS.

### EXAMPLE 1: SYNTHESIS OF PEG-LINKER 1H

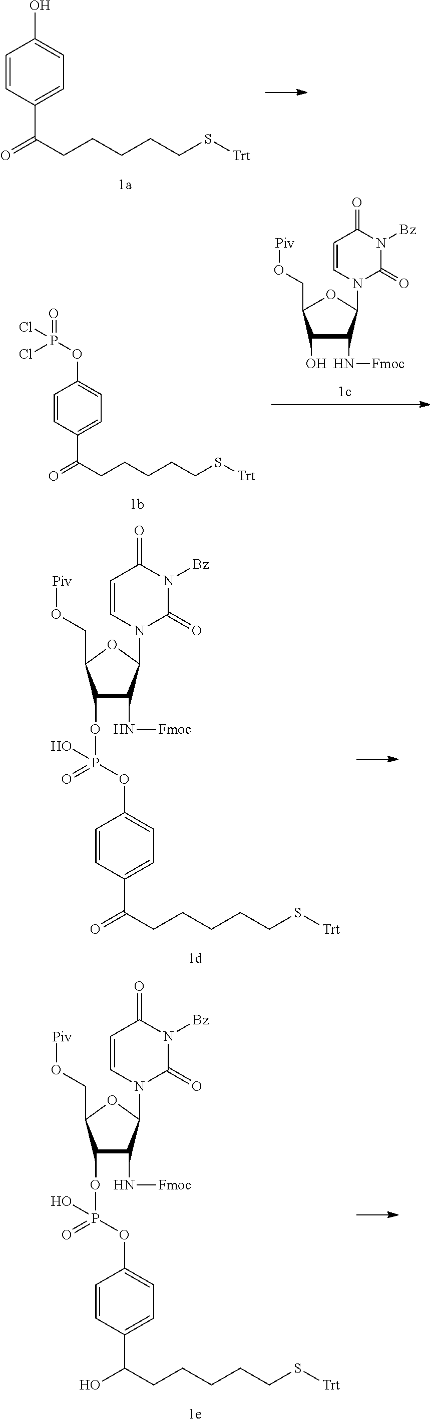

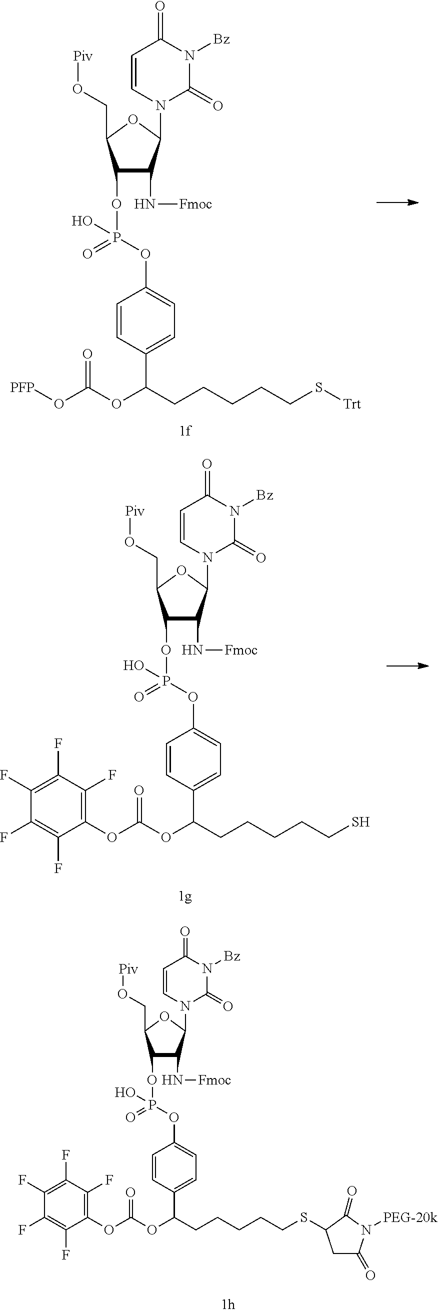

Compound 1a is synthesized as described in WO 2005/099768 A2 for compound 55b.

In an atmosphere of argon, a solution of triethylamine (1 eq.) in diethyl ether is added dropwise to a solution of 1a (1 eq.) and freshly distilled phosphoryl chloride (3 eq.) in diethyl ether under cooling in an ice-bath. After 1 h at 0° C. the reaction is stirred for 2 h at room temperature. The reaction is filtered, and the filtrate is concentrated in vacuo. Diethyl ether is added to the residue, the mixture filtered, and the filtrate is concentrated in vacuo. Compound 1b is used without further purification.

Compound 1c is synthesized as described in WO 2020/053815 A1 in example 8.

Under an atmosphere of argon, a solution of 1b (2 eq.) in acetonitrile is added dropwise to a solution of 1c (1 eq.) in pyridine while stirring. After 2 h the reaction is quenched with 1 M triethylammonium bicarbonate solution and extracted with DCM. The organic phase is concentrated and coevaporated with toluene. The product 1d is purified on silica gel using a gradient of methanol in DCM and 0.1% pyridine. The product 1d is isolated as the triethylammonium salt.

Compound 1d (1 eq.) is dissolved in MeOH and NaBH₄ (5 eq.) is added. The reaction is stirred for 30 min once the evolution of gas has ceased. The reaction is diluted with 0.1 M triethylammonium acetate buffer and purified by RP-HPLC with a gradient of acetonitrile in 0.1 M triethylammonium buffer. The product 1e is isolated as the triethylammonium salt.

Compound 1e (1 eq.) is dissolved in acetonitrile and the solution cooled with an ice-bath. Bis(PFP)carbonate (2.5 eq) is added then DMAP (0.25 eq.) and triethylamine (5 eq.) are added. The reaction is stirred for 2 h. The product 1f is purified by RP-HPLC with a gradient of acetonitrile in water+0.10% TFA.

1f (1 eq.) is stirred in HFIP/acetic acid/TES 95:5:5. After 30 min the reaction is diluted with water and the HFIP removed by concentration in vacuo and the product 1g purified by RP-HPLC with a gradient of acetonitrile in water+0.10% TFA.

1g (1 eq.) is dissolved in acetonitrile/water 9:1 and cooled in an ice-bath. mPEG-20 kDa-maleimide (1.2 eq.) is dissolved and sodium phosphate buffer (0.25 M, pH 7.2) is added until the pH reaches 6-7. After 30 min the product 1f is purified by RP-HPLC with a gradient of acetonitrile in water+0.1% TFA.

### EXAMPLE 2: SYNTHESIS OF PEG-LINKER 2J

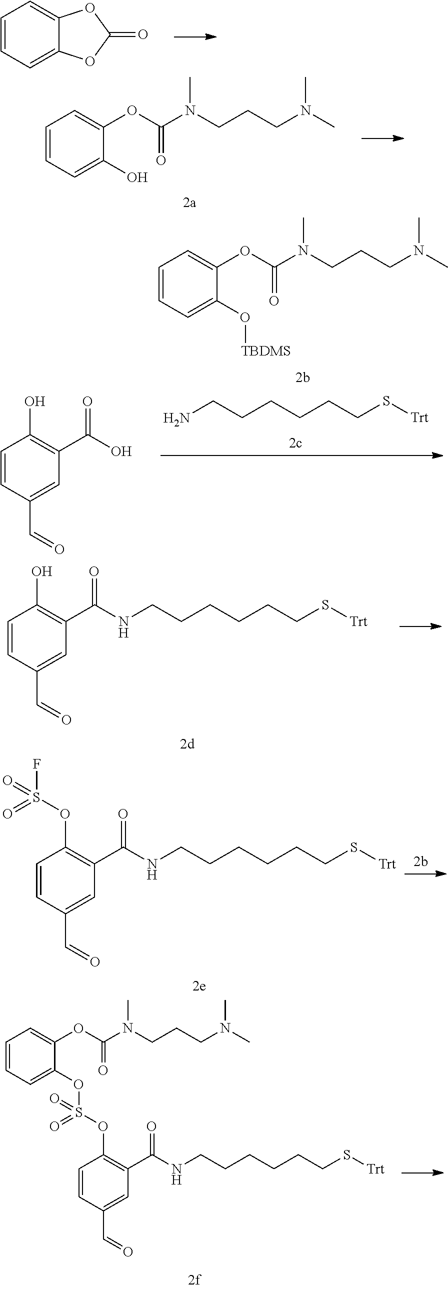

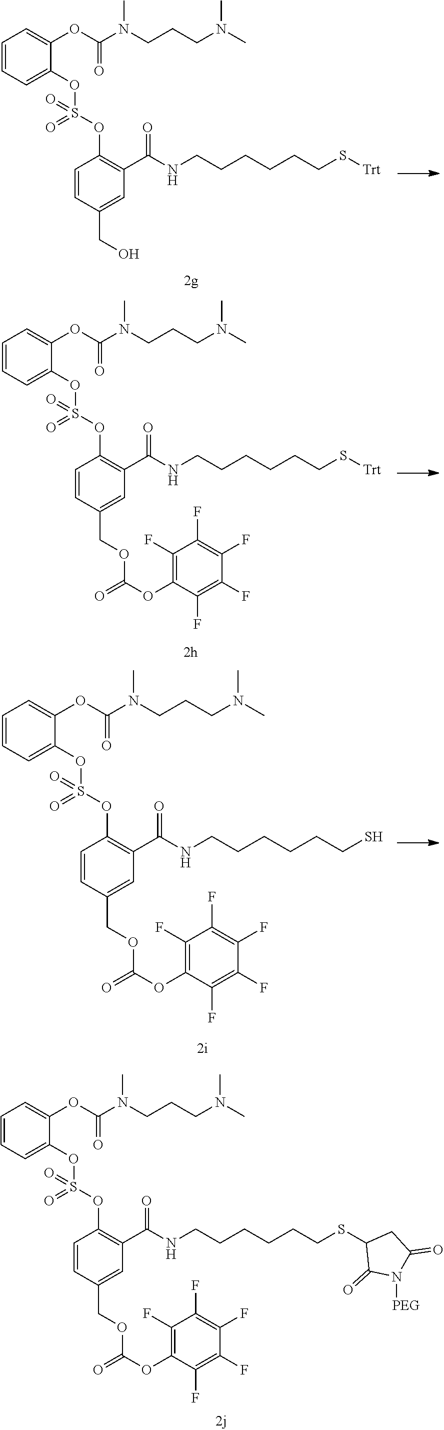

1,3-benzodioxol-2-one (1 eq.) and N,N,N′-trimethyl-1,3-propanediamine (1.05 eq.) are stirred for 1 h at room temperature in THF. Then the solution is cooled in an ice-bath and triethylamine (1.3 eq.) and TBDMSCl (1.3 eq.) are added. After 2 h stirring at 0° C. the reaction is diluted with DCM and water. The organic phase is dried (MgSO₄), concentrated in vacuo, and the product is purified by flash chromatography on silica gel yielding 2b.

Compound 2c is synthesized as described in WO 2016/020373 A1 for compound 34.

5-Formyl-2-hydroxybenzoic acid (1.1 eq.), 2c (1 eq.), HOBt (1.1 eq) and DIPEA (1.5 eq.) are dissolved in DMF, and DIC (1.1 eq.) is added with stirring. After 16 h the reaction is diluted with ethyl acetate and washed with sat. sodium bicarbonate solution and brine. The organic phase is dried (MgSO₄), concentrated in vacuo, and the product is purified by flash chromatography on silica gel yielding 2d.

2d (1 eq.) is dissolved in DCM and triethylamine (2 eq.) is added under an atmosphere of nitrogen. SO₂F₂ gas is introduced to the reaction via balloon. After 2 h the reaction is diluted with DCM and washed with sat. sodium bicarbonate solution and brine. The organic phase is dried (MgSO₄), concentrated in vacuo, and the product is purified by flash chromatography on silica gel yielding 2e.

2e (1 eq.) and 2b (1 eq.) are combined in acetonitrile and DBU (0.2 eq.) is added. After 1 h the reaction is quenched with TFA and purified by RP-HPLC with a gradient of acetonitrile in water+0.1% TFA yielding 2f as the TFA salt.

2f (1 eq.) is dissolved in THF and NaBH₄ (2 eq.) is added at 0° C. The reaction is stirred for 2 h at room temperature. The reaction is quenched with 10% aq TFA and the product purified by RP-HPLC with a gradient of acetonitrile in water+0.1% TFA yielding 2g as the TFA salt.

Compound 2g (1 eq.) is dissolved in acetonitrile and the solution cooled with an ice-bath. Bis(PFP)carbonate (2.5 eq.) is added then DMAP (0.25 eq.) and triethylamine (5 eq.) are added. The reaction is stirred for 2 h. The product 2h is purified by RP-HPLC with a gradient of acetonitrile in water+0.10% TFA.

2h (1 eq.) is stirred in HFIP/acetic acid/TES 95:5:5. After 30 min the reaction is diluted with water and the HFIP removed by concentration in vacuo and the product 2i purified by RP-HPLC with a gradient of acetonitrile in water+0.10% TFA.

2i (1 eq.) is dissolved in acetonitrile/water 9:1 and cooled in an ice-bath. mPEG-20 kDa-maleimide (1.2 eq.) is dissolved and sodium phosphate buffer (0.25 M, pH 7.2) is added until the pH reaches 6-7. After 30 min the product 2j is purified by RP-HPLC with a gradient of acetonitrile in water+0.1% TFA.

### EXAMPLE 3

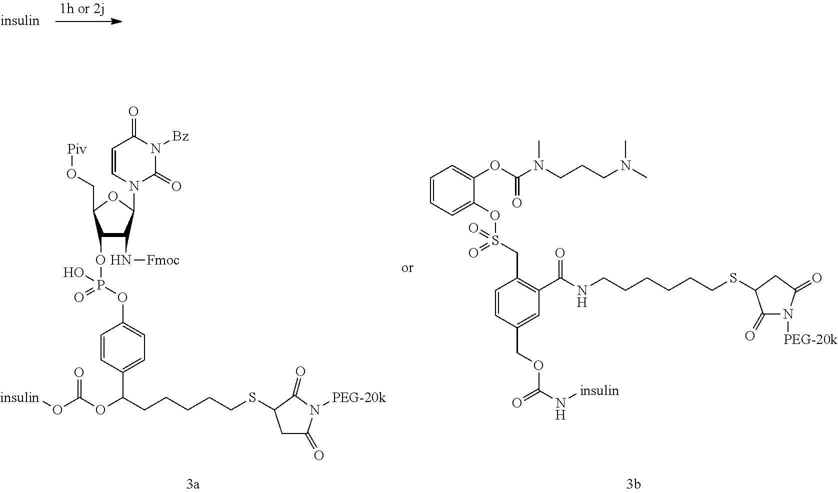

Insulin (1 eq.) is dissolved in a mixture of 0.375 M sodium borate buffer pH 8.5 and DMF 4:6(v/v) and activated PEG linker 1h or 2j (1.1 eq.) is added. The mono pegylated products are purified by RP-HPLC with a gradient of acetonitrile in water+0.1% TFA.

### Abbreviations

Bz benzoyl
DBU 1,8-diazabicyclo (5.4.0)undec-7-ene
DCM dichloromethane
DIC N,N′-diisopropylcarbodiimide
DIPEA diisopropylethylamine
DMAP dimethylaminopyridine
DMF dimethylformamide
eq. equivalent
Fmoc fluorenylmethyloxycarbonyl
HFIP 1,1,1,3,3,3-hexafluoroisopropanol
HOBt 1-hydroxybenzotriazole
HPLC high performance liquid chromatography
LCMS liquid chromatography mass spectrometry
MeOH methanol
PEG polyethylene glycol
PFP pentafluorophenyl
Piv pivaloyl
RP reversed phase
sat. saturated
TBDMS tert. butyldimethylsilyl
TES triethylsilane
TFA trifluoroacetic acid
THF tetrahydrofurane
Trt trityl

1. A conjugate or a pharmaceutically acceptable salt thereof comprising at least one moiety
-D, conjugated via at least one moiety -L¹-L²- to at least one moiety Z, wherein a moiety -L¹- is conjugated to a moiety -D and wherein the linkage between -D and -L¹- is reversible and wherein a moiety -L²- is conjugated to Z, wherein
each -D is independently a drug moiety;
each -L²- is independently a single bond or a spacer moiety;
each Z is independently a polymeric moiety or a C₈₋₂₄ alkyl;
each -L¹- is independently a linker moiety of formula (I): 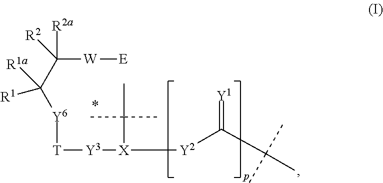
wherein
the dashed line marked with an asterisk indicates the attachment to -L²-;
the unmarked dashed line indicates the attachment to -D;
p is selected from the group consisting of 0 and 1;
—W— is selected from the group consisting of —O— and —N(R³)—;
-E is a cleavable moiety;
-T- is selected from the group consisting of 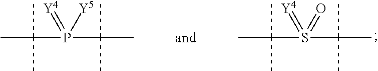
═Y¹ and ═Y⁴ are independently selected from the group consisting of ═O, ═S and ═N(R⁴);
—Y²—, —Y³— and —Y⁶— are independently selected from the group consisting of —O—, —S— and —N(R⁴)—;
—Y⁵ is selected from the group consisting of —O(R⁵), —S(R⁵) and —N(R⁴)(R^{4a});
—X— is selected from the group consisting of:
C₂₋₃ alkyl, 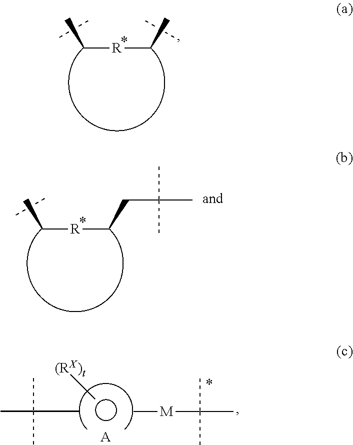
provided that if —X— is C₂₋₃ alkyl, of formula (a) or (b) then p is 1;
—R*— is selected from the group consisting of C₃₋₁₀ cycloalkyl and 3- to 10-membered heterocyclyl;
wherein in formula (c) the unmarked dashed line indicates the attachment point to —Y³— and the marked dashed line indicates the attachment to —Y²— if p is 1 or to -D if p is 0 and wherein -A- is a ring selected from the group consisting of mono-, bi- or tricylic aryl and heteroaryl, provided that -A- is connected to —Y³— and -M- via carbon atoms; wherein said mono-, bi- or tricyclic aryl and heteroaryl is optionally substituted with one or more —R^{x}, which are the same or different;
t is selected from the group consisting of 0, 1, 2, 3, 4, 5, 6, 7, 8, 9 and 10;
wherein -M- is selected from the group consisting of formula (i) and (ii): 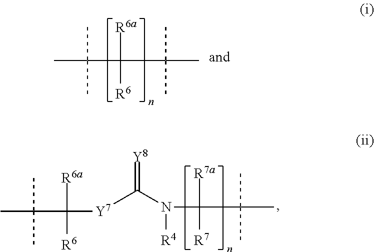
—Y⁷— is selected from the group consisting of —O—, —S— and —N(R⁴)—;
=Y⁸ is selected from the group consisting of ═O, ═S and ═N(R⁴);
n is independently selected from the group consisting of 1, 2 and 3;
—R¹, —R^{1a}, —R², —R^{2a} are independently selected from the group consisting of —H, —C(O)OH, halogen, —NO₂, —CN, —OH, -T*, C₁₋₆ alkyl, C₂₋₆ alkenyl and C₂₋₆ alkynyl;
wherein C₁₋₆ alkyl, C₂₋₆ alkenyl and C₂₋₆ alkynyl are optionally substituted with one or more —R^{x}, which are the same or different; and wherein C₁₋₆ alkyl, C₂₋₆ alkenyl and C₂₋₆ alkynyl are optionally interrupted by one or more groups selected from the group consisting of -T*-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
—R⁵, each —R⁶, each —R^{6a}, each —R⁷ and each —R^{7a} are independently selected from the group consisting of —H, -T*, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{x}, which are the same or different; and wherein C₁₋₆ alkyl, C₂₋₆ alkenyl and C₂₋₆ alkynyl are optionally interrupted by one or more groups selected from the group consisting of -T*-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
each T* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T* is independently optionally substituted with one or more —R^{x}, which are the same or different;
each —R^{x} is independently selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
wherein —R³, —R⁴ and —R^{4a} are independently selected from the group consisting of —H and C₁₋₆ alkyl; wherein C₁₋₆ alkyl is optionally substituted with one or more halogen, which are the same or different;
optionally, —R¹ and —R² are joined together to form a carbohydrate-nucleobase system;
optionally, —R¹ and —R² are joined together with the carbon atoms to which they are attached to form a ring T*;
optionally, —R³ and —R² together with the nitrogen to which —R³ is attached and the carbon atom to which —R² is attached form a 3- to 10-membered heterocyclyl or an 8- to 11-membered heterobicyclyl, which 3- to 10-membered heterocyclyl or 8- to 11-membered heterobicyclyl is optionally substituted with one or more —R^{x} which are the same or different;
optionally, —R³ and —R¹ together with the nitrogen to which —R³ is attached, the carbon atom to which —R² is attached and the carbon atom to which —R¹ is attached form a 3- to 10-membered heterocyclyl or an 8- to 11-membered heterobicyclyl, which 3- to 10-membered heterocyclyl or 8- to 11-membered heterobicyclyl is optionally substituted with one or more —R^{x} which are the same or different;
optionally, —R⁶ and an adjacent —R^{6a} of formula (i) or —R⁷ and an adjacent —R⁷ of formula (ii) form a carbon-carbon double bond provided that n is selected from the group consisting of 2 and 3; and
wherein each -L¹- is substituted with -L²- and optionally further substituted.

2. The conjugate or pharmaceutically acceptable salt thereof of claim 1, wherein -T- is

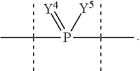

3. The conjugate or pharmaceutically acceptable salt thereof of claim 1, wherein -T- is 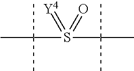
and ═Y⁴ is ═O.

4. The conjugate or pharmaceutically acceptable salt thereof of claim 1, wherein —W— is —O—.

5. The conjugate or pharmaceutically acceptable salt thereof of claim 1, wherein —R¹ and —R² are joined together with the carbon atoms to which they are attached to form a ring T*.

6. The conjugate or pharmaceutically acceptable salt thereof of claim 1, wherein -E is of formula (a′): 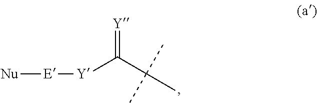
wherein the dashed line indicates the attachment to —W—;
-Nu is a nucleophile;
-E′- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R^{x}, which are the same or different;
-Q- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -Q- is independently optionally substituted with one or more —R^{x}, which are the same or different;
—Y′— is selected from the group consisting of —O—, —C(R⁶)(R^{6a})—, —N(R⁴)— and —S—;
═Y″ is selected from the group consisting of ═O, ═S and ═N(R⁴); and
—R^{x}, —R⁴, —R⁶ and —R^{6a} are used as defined in claim 1.

7. The conjugate or pharmaceutically acceptable salt thereof of claim 6, wherein -Nu of formula (a′) is selected from the group consisting of primary, secondary or tertiary amine and amide.

8. The conjugate or pharmaceutically acceptable salt thereof of claim 6, wherein -E′- of formula (a′) is selected from the group consisting of C₁₋₆ alkyl, C₂₋₆ alkenyl and C₂₋₆ alkynyl.

9. The conjugate or pharmaceutically acceptable salt thereof of claim 1, wherein -L¹- is of formula (I′a): 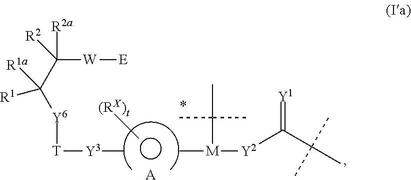
wherein the dashed line marked with the asterisk indicates the attachment to -L²- of formula (I) and the unmarked dashed line indicates the attachment to -D of formula (I);
t is selected from the group consisting of 1, 2, 3, 4, 5, 6, 7, 8, 9 and 10; and
═Y¹, —Y²—, -M-, —Y³—, -A-, -T-, —Y⁶—, —W—, -E, —R¹, —R^{1a}, —R², —R^{2a} and —R^{x} are used as defined in claim 1.

10. The conjugate or pharmaceutically acceptable salt thereof of claim 1, wherein t is selected from the group consisting of 1, 2, 3, 4, 5, 6, 7 and 8.

11. The conjugate or pharmaceutically acceptable salt thereof of claim 1, wherein -L¹- is of formula (I′d): 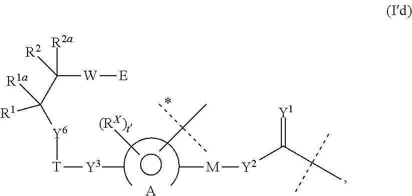
wherein the dashed line marked with the asterisk indicates the attachment to -L²- of formula (I) and the unmarked dashed line indicates the attachment to -D of formula (I);
t′ is selected from the group consisting of 1, 2, 3, 4, 5, 6, 7, 8 and 9; and
═Y¹, —Y²—, -M-, -A-, —Y³—, -T-, —Y⁶—, —W—, -E, —R¹, —R^{1a}, —R², —R^{2a} and —R^{x} are used as defined in claim 1.

12. The conjugate or pharmaceutically acceptable salt thereof of claim 11, wherein t′ is selected from the group consisting of 1, 2, 3, 4, 5, 6 and 7.

13. The conjugate or pharmaceutically acceptable salt thereof of claim 1, wherein —X— is of formula (c), -M- is of formula (i): 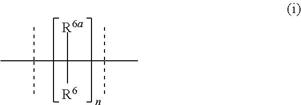
and n is independently selected from the group consisting of 1, 2 and 3.

14. The conjugate or pharmaceutically acceptable salt thereof of claim 13, wherein n is 1.

15. The conjugate or pharmaceutically acceptable salt thereof of claim 13, wherein —R⁶ and —R^{6a} are both —H.

16. The conjugate or pharmaceutically acceptable salt thereof of claim 1, wherein —X— is of formula (c), -A- is a ring selected from the group consisting of monocyclic or bicyclic aryl and heteroaryl and t is selected from the group consisting of 0, 1, 2, 3, 4, 5 and 6.

17. The conjugate or pharmaceutically acceptable salt thereof of claim 1, wherein —X— is of formula (c) and -A- is selected from the group consisting of 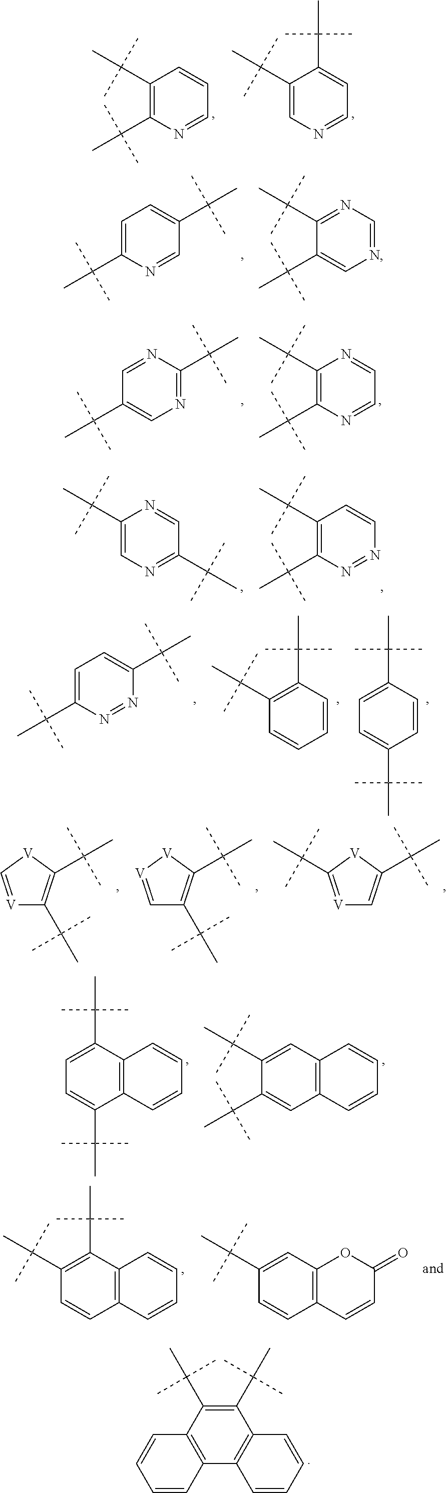
wherein each V is independently selected from the group consisting of O, S and N.

18. The conjugate or pharmaceutically acceptable salt thereof of claim 1, wherein Z is a polymeric moiety.

19. The conjugate or pharmaceutically acceptable salt thereof of claim 1, wherein Z is a water-insoluble polymeric moiety.

20. The conjugate or pharmaceutically acceptable salt thereof of claim 1, wherein Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

21. The conjugate or pharmaceutically acceptable salt thereof of claim 1, wherein Z is a hydrogel.

22. The conjugate or pharmaceutically acceptable salt thereof of claim 1, wherein Z is a PEG-based or hyaluronic-acid based hydrogel.

23. The conjugate or pharmaceutically acceptable salt thereof of claim 1, wherein Z is a water-soluble polymeric moiety.

24. The conjugate or pharmaceutically acceptable salt thereof of claim 1, wherein -D is selected from the group consisting of small molecule, medium size molecule, oligonucleotide, peptide nucleic acid, peptide and protein drug moieties.

25. The conjugate or pharmaceutically acceptable salt thereof of claim 1, wherein -L²- is a spacer moiety.

26. A pharmaceutical composition comprising the conjugate or pharmaceutically acceptable salt thereof of claim 1.

27. A method of treating a disease comprising administering a therapeutically effective amount of the conjugate or pharmaceutically acceptable salt thereof of claim 1.

28. The method of claim 27 wherein the disease can be treated with D-H.
